SEQUENCE LISTING (59 sequences)

SEQ ID NO: 1 - protein, 441 aa, Artificial Sequence
  Modified human antibody chain
  THIOETH (373)..(373): Xaa at position 373 is cysteine residue modified by thioether bond formation to maleimide-PEG linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIXVEWESNGQPENNYKTTPPVLDSDGSFFLYSRLTVDKSRWQEGNVFSCSVMHEALHNHYTQKSLSLSLG

SEQ ID NO: 2 - protein, 441 aa, Artificial Sequence
  Modified human antibody chain
  THIOETH (122)..(122): Xaa at position 122 is cysteine residue modified by thioether bond formation to maleimide-PEG linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPXVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 3 - protein, 441 aa, Artificial Sequence
  Modified human antibody chain
  THIOETH (122)..(122): Xaa at position 122 is cysteine residue modified by thioether bond formation to maleimide-PEG linker
  THIOETH (373)..(373): Xaa at position 373 is cysteine residue modified by thioether bond formation to maleimide-PEG linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPXVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIXVEWESNGQPENNYKTTPPVLDSDGSFFLYSRLTVDKSRWQEGNVFSCSVMHEALHNHYTQKSLSLSLG

SEQ ID NO: 4 - protein, 441 aa, Artificial Sequence
  Modified human antibody chain
  THIOETH (122)..(122): Xaa at position 122 is cysteine residue modified by thioether bond formation to maleimide-PEG linker
  THIOETH (370)..(370): Xaa at position 370 is cysteine residue modified by thioether bond formation to maleimide-PEG linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPXVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPXDIAVEWESNGQPENNYKTTPPVLDSDGSFFLYSRLTVDKSRWQEGNVFSCSVMHEALHNHYTQKSLSLSLG

SEQ ID NO: 5 - protein, 215 aa, Homo sapiens
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 449 aa, Artificial Sequence
  Modified human antibody chain
  THIOETH (127)..(127): Xaa at position 127 is cysteine residue modified by thioether bond formation to maleimide-PEG linker
  THIOETH (381)..(381): Xaa at position 381 is cysteine residue modified by thioether bond formation to maleimide-PEG linker
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPXVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIXVEWESNGQPENNYKTTPPVLDSDGSFFLYSKLTVDKSRWQQGNVFSCSVMHEALHNHYTQKSLSLSPG

SEQ ID NO: 7 - protein, 214 aa, Homo sapiens
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 450 aa, Artificial Sequence
  Modified human antibody chain
  THIOETH (128)..(128): Xaa at position 128 is cysteine residue modified by thioether bond formation to maleimide-PEG linker
  THIOETH (382)..(382): Xaa at position 382 is cysteine residue modified by thioether bond formation to maleimide-PEG linker
EVQLVQSGAEVKKPGESLKISCKGSGRTFTSYNMHWVRQMPGKGLEWMGAIYPLTGDTSYNQKSKLQVTISADKSISTAYLQWSSLKASDTAMYYCARSTYVGGDWQFDVWGKGTTVTVSSASTKGPXVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKIKDTLMISRTPEVTCVVVDVSHEDPEVKFNWYVDGVEVHNAKTKPREEQYNSTYRVVSVLTVLHQDWLNGKEYKCKVSNKALPAPIEKTISKQKGQPREPQVYTLPPSRDELTKNQVSLTCLVKGFYPSDIXVEWESNGQPENNYKTTPPVLDSDGSFFLYSKLTVDKSRWQQGNVFSCSVMHEALHNHYTQKSLSLSPG

SEQ ID NO: 9 - protein, 213 aa, Homo sapiens
EIVLTQSPGTLSLSPGERATLSCRASSSVPYIHWYQQKPGQAPRLLIYATSALASGIPDRFSGSGSGTDFTLTISRLEPEDFAVYYCQQWLSNPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 326 aa, Artificial Sequence
  Modified human antibody chain
  THIOETH (7)..(7): Xaa at position 7 is cysteine residue modified by thioether bond formation to maleimide-PEG linker
ASTKGPXVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 13 - protein, 326 aa, Artificial Sequence
  Modified human antibody chain
  THIOETH (258)..(258): Xaa at position 258 is cysteine residue modified by thioether bond formation to maleimide-PEG linker
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIXVEWESNGQPENNYKTTPPVLDSDGSFFLYSRLTVDKSRWQEGNVFSCSVMHEALHNHYTQKSLSLSLG

SEQ ID NO: 14 - protein, 326 aa, Artificial Sequence
  Modified human antibody chain
  THIOETH (255)..(255): Xaa at position 255 is cysteine residue modified by thioether bond formation to maleimide-PEG linker
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPXDIAVEWESNGQPENNYKTTPPVLDSDGSFFLYSRLTVDKSRWQEGNVFSCSVMHEALHNHYTQKSLSLSLG

SEQ ID NO: 15 - protein, 326 aa, Artificial Sequence
  Modified human antibody chain
  THIOETH (7)..(7): Xaa at position 7 is cysteine residue modified by thioether bond formation to maleimide-PEG linker
  THIOETH (258)..(258): Xaa at position 258 is cysteine residue modified by thioether bond formation to maleimide-PEG linker
ASTKGPXVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIXVEWESNGQPENNYKTTPPVLDSDGSFFLYSRLTVDKSRWQEGNVFSCSVMHEALHNHYTQKSLSLSLG

SEQ ID NO: 16 - protein, 326 aa, Artificial Sequence
  Modified human antibody chain
  THIOETH (7)..(7): Xaa at position 7 is cysteine residue modified by thioether bond formation to maleimide-PEG linker
  THIOETH (255)..(255): Xaa at position 255 is cysteine residue modified by thioether bond formation to maleimide-PEG linker
ASTKGPXVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPXDIAVEWESNGQPENNYKTTPPVLDSDGSFFLYSRLTVDKSRWQEGNVFSCSVMHEALHNHYTQKSLSLSLG

SEQ ID NO: 17 - protein, 330 aa, Artificial Sequence
  Modified human antibody chain
  THIOETH (7)..(7): Xaa at position 7 is cysteine residue modified by thioether bond formation to maleimide-PEG linker
ASTKGPXVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 330 aa, Artificial Sequence
  Modified human antibody chain
  THIOETH (261)..(261): Xaa at position 261 is cysteine residue modified by thioether bond formation to maleimide-PEG linker
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIXVEWESNGQPENNYKTTPPVLDSDGSFFLYSKLTVDKSRWQQGNVFSCSVMHEALHNHYTQKSLSLSPGK

SEQ ID NO: 19 - protein, 330 aa, Artificial Sequence
  Modified human antibody chain
  THIOETH (258)..(258): Xaa at position 258 is cysteine residue modified by thioether bond formation to maleimide-PEG linker
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPXDIAVEWESNGQPENNYKTTPPVLDSDGSFFLYSKLTVDKSRWQQGNVFSCSVMHEALHNHYTQKSLSLSPGK

SEQ ID NO: 20 - protein, 330 aa, Artificial Sequence
  Modified human antibody chain
  THIOETH (7)..(7): Xaa at position 7 is cysteine residue modified by thioether bond formation to maleimide-PEG linker
  THIOETH (261)..(261): Xaa at position 261 is cysteine residue modified by thioether bond formation to maleimide-PEG linker
ASTKGPXVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIXVEWESNGQPENNYKTTPPVLDSDGSFFLYSKLTVDKSRWQQGNVFSCSVMHEALHNHYTQKSLSLSPGK

SEQ ID NO: 21 - protein, 330 aa, Artificial Sequence
  Modified human antibody chain
  THIOETH (7)..(7): Xaa at position 7 is cysteine residue modified by thioether bond formation to maleimide-PEG linker
  THIOETH (258)..(258): Xaa at position 258 is cysteine residue modified by thioether bond formation to maleimide-PEG linker
ASTKGPXVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPXDIAVEWESNGQPENNYKTTPPVLDSDGSFFLYSKLTVDKSRWQQGNVFSCSVMHEALHNHYTQKSLSLSPGK

SEQ ID NO: 22 - protein, 4 aa, Artificial Sequence
  N-formylated peptide
  MOD_RES (1)..(1): FORMYLATION
  MISC_FEATURE (4)..(4): Xaa at position 4 is lysine residue modified by amide bond formation to maleimide-PEG linker
MLFX

SEQ ID NO: 23 - protein, 4 aa, Artificial Sequence
  N-formylated peptide
  MOD_RES (1)..(1): FORMYLATION
MLFK

SEQ ID NO: 24 - protein, 4 aa, Artificial Sequence
  Peptide with maleimido-PEG linker
  MISC_FEATURE (4)..(4): Xaa at position 4 is lysine residue modified by amide bond formation to maleimide-PEG linker
MLFX

SEQ ID NO: 25 - protein, 4 aa, Artificial Sequence
  Peptide without N-formylation
MLFK

SEQ ID NO: 26 - protein, 441 aa, Artificial Sequence
  Modified human antibody chain
  THIOETH (410)..(410): Xaa at position 410 is cysteine residue modified by thioether bond formation to maleimide-PEG linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKXRWQEGNVFSCSVMHEALHNHYTQKSLSLSLG

SEQ ID NO: 27 - protein, 215 aa, Artificial Sequence
  Modified human antibody chain
  THIOETH (157)..(157): Xaa at position 157 is cysteine residue modified by thioether bond formation to maleimide-PEG linker
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQXGNSQESVTEQDSKDSTYSLSSTLTLSKADYEKHKVYACEVTHQGLSSPVTKSFNRGEC

SEQ ID NO: 28 - protein, 215 aa, Artificial Sequence
  Modified human antibody chain
  THIOETH (172)..(172): Xaa at position 172 is cysteine residue modified by thioether bond formation to maleimide-PEG linker
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDXTYSLSSTLTLSKADYEKHKVYACEVTHQGLSSPVTKSFNRGEC

SEQ ID NO: 29 - protein, 215 aa, Artificial Sequence
  Modified human antibody chain
  THIOETH (192)..(192): Xaa at position 192 is cysteine residue modified by thioether bond formation to maleimide-PEG linker
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKXYACEVTHQGLSSPVTKSFNRGEC

SEQ ID NO: 30 - protein, 215 aa, Artificial Sequence
  Modified human antibody chain
  THIOETH (194)..(194): Xaa at position 194 is cysteine residue modified by thioether bond formation to maleimide-PEG linker
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYXCEVTHQGLSSPVTKSFNRGEC

SEQ ID NO: 31 - protein, 215 aa, Artificial Sequence
  Modified human antibody chain
  THIOETH (203)..(203): Xaa at position 203 is cysteine residue modified by thioether bond formation to maleimide-PEG linker
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLXSPVTKSFNRGEC

SEQ ID NO: 32 - protein, 215 aa, Artificial Sequence
  Modified human antibody chain
  THIOETH (209)..(209): Xaa at position 209 is cysteine residue modified by thioether bond formation to maleimide-PEG linker
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKXFNRGEC

SEQ ID NO: 33 - protein, 449 aa, Artificial Sequence
  Modified human antibody chain
  THIOETH (127)..(127): Xaa at position 127 is cysteine residue modified by thioether bond formation to maleimide-PEG linker
  THIOETH (160)..(160): Xaa at position 160 is cysteine residue modified by thioether bond formation to maleimide-PEG linker
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPXVFPLAPSSKSTSGGTAALGCLVKDYFPEPVTVXWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 448 aa, Artificial Sequence
  Modified human antibody chain
  THIOETH (126)..(126): Xaa at position 126 is cysteine residue modified by thioether bond formation to maleimide-PEG linker
  THIOETH (380)..(380): Xaa at position 380 is cysteine residue modified by thioether bond formation to maleimide-PEG linker
EVQLVESGGGLVQPGGSLRLSCAASGFTFTDYTMDWVRKAPGKGLEWVADVNPNSGGSIYNQEFKGRFTLSVDRSKNTLYLQMNSLRAEDTAVYYCARNLGPSFYFDYWGQGTLVTVSSASTKGPXVFPLAPSSKSTSGGTAALGCLVKDYFPEPVTVSWNSGALTSGVATGPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIXVEWESNGQPENNYDTTPPVLDSDGSFFLYSDLTVDKSRWQQGNVFSCSVMHEALHNHYTQKSLSLSPG

SEQ ID NO: 35 - protein, 450 aa, Artificial Sequence
  Modified human antibody chain
  THIOETH (128)..(128): Xaa at position 128 is cysteine residue modified by thioether bond formation to maleimide-PEG linker
  THIOETH (382)..(382): Xaa at position 382 is cysteine residue modified by thioether bond formation to maleimide-PEG linker
QVQLVQSGAEVKKPGASVKVSCKASGYTFTSHWMHWVRYAPGQGLEWIGEFNPSNGRTNYNEKFKSKATMTVDTSTNTAYMELSSLRSEDTAVYYCASRDYDYDGRYFDYWGQGTLVTVSSASTKGPXVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRKELTKNQVSLTCLVKGFYPSDIXVEWESNGQPENNYKTTPPVLKSDGSFFLYSKLTVDKSRWQQGNVFSCSVMHEALHNHYTQKSLSLSPG

SEQ ID NO: 36 - protein, 5 aa, Artificial Sequence
  N-formylated peptide with maleimido-PEG linker
  MOD_RES (1)..(1): FORMYLATION
  MISC_FEATURE (5)..(5): Xaa at position 5 is lysine residue side chain modified through epsilon amide bond formation to a hydrolyzed maleimide-PEG linker
MIFLX

SEQ ID NO: 37 - protein, 4 aa, Artificial Sequence
  N-formylated peptide
  MOD_RES (1)..(1): FORMYLATION
  MISC_FEATURE (2)..(2): Xaa at position 2 is diethylglycine
  MISC_FEATURE (4)..(4): Xaa at position 4 is leucine residue C-terminally connected by amide bond formation to a PEG linker
MXFX

SEQ ID NO: 38 - protein, 4 aa, Artificial Sequence
  N-formylated peptide
  MOD_RES (1)..(1): FORMYLATION
  MISC_FEATURE (2)..(2): Xaa at position 2 is dipropylglycine
  MISC_FEATURE (4)..(4): Xaa at position 4 is leucine residue C-terminally connected by amide bond formation to a PEG linker
MXFX

SEQ ID NO: 39 - protein, 3 aa, Artificial Sequence
  N-formylated peptide
  MOD_RES (1)..(1): FORMYLATION
  MISC_FEATURE (3)..(3): Xaa at position 3 is phenylalanine residue C-terminally connected by amide bond formation to a PEG linker
MIX

SEQ ID NO: 40 - protein, 4 aa, Artificial Sequence
  N-formylated peptide
  MOD_RES (1)..(1): FORMYLATION
  MISC_FEATURE (4)..(4): Xaa at position 4 is leucine residue C-terminally connected by amide bond formation to a PEG linker
MIFX

SEQ ID NO: 41 - protein, 4 aa, Artificial Sequence
  N-formylated peptide
  MOD_RES (1)..(1): FORMYLATION
  MISC_FEATURE (4)..(4): Xaa at position 4 is leucine residue modified by amide bond formation to a maleimide-PEG linker
MIFX

SEQ ID NO: 42 - protein, 3 aa, Artificial Sequence
  N-formylated peptide
  MOD_RES (1)..(1): FORMYLATION
  MISC_FEATURE (1)..(1): Xaa at position 1 is norleucine
  MISC_FEATURE (3)..(3): Xaa at position 3 is phenylalanine C-terminally connected by amide bond formation to a maleimide-PEG linker
XLX

SEQ ID NO: 43 - protein, 441 aa, Artificial Sequence
  Modified human antibody chain
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 44 - protein, 441 aa, Artificial Sequence
  Modified human antibody chain
  THIOETH (155)..(155): Xaa at position 155 is cysteine residue modified by thioether bond formation to maleimide-PEG linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVXWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 45 - protein, 441 aa, Artificial Sequence
  Modified human antibody chain
  THIOETH (160)..(160): Xaa at position 160 is cysteine residue modified by thioether bond formation to maleimide-PEG linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGXLTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 46 - protein, 441 aa, Artificial Sequence
  Modified human antibody chain
  THIOETH (257)..(257): Xaa at position 257 is cysteine residue modified by thioether bond formation to maleimide-PEG linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCXVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 47 - protein, 441 aa, Artificial Sequence
  Modified human antibody chain
  THIOETH (370)..(370): Xaa at position 370 is cysteine residue modified by thioether bond formation to maleimide-PEG linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPXDIAVEWESNGQPENNYKTTPPVLDSDGSFFLYSRLTVDKSRWQEGNVFSCSVMHEALHNHYTQKSLSLSLG

SEQ ID NO: 48 - protein, 441 aa, Artificial Sequence
  Modified human antibody chain
  THIOETH (392)..(392): Xaa at position 392 is cysteine residue modified by thioether bond formation to maleimide-PEG linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPXLDSDGSFFLYSRLTVDKSRWQEGNVFSCSVMHEALHNHYTQKSLSLSLG

SEQ ID NO: 49 - protein, 441 aa, Artificial Sequence
  Modified human antibody chain
  MISC_FEATURE (122)..(122): Xaa at position 122 is cysteine residue modified by thioether bond formation to maleimide-PEG linker
  MISC_FEATURE (155)..(155): Xaa at position 155 is cysteine residue modified by thioether bond formation to maleimide-PEG linker
  MISC_FEATURE (373)..(373): Xaa at position 373 is cysteine residue modified by thioether bond formation to maleimide-PEG linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPXVFPLAPCSRSTSESTAALGCLVKDYFPEPVTVXWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIXVEWESNGQPENNYKTTPPVLDSDGSFFLYSRLTVDKSRWQEGNVFSCSVMHEALHNHYTQKSLSLSLG

SEQ ID NO: 50 - protein, 441 aa, Artificial Sequence
  Modified human antibody chain
  THIOETH (122)..(122): Xaa at position 122 is cysteine residue modified by thioether bond formation to maleimide-PEG linker
  THIOETH (160)..(160): Xaa at position 160 is cysteine residue modified by thioether bond formation to maleimide-PEG linker
  THIOETH (373)..(373): Xaa at position 373 is cysteine residue modified by thioether bond formation to maleimide-PEG linker
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPXVFPLAPCSRSTSESTAALGCLVKDYFPEPVTVSWNSGXLTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIXVEWESNGQPENNYKTTPPVLDSDGSFFLYSRLTVDKSRWQEGNVFSCSVMHEALHNHYTQKSLSLSLG

SEQ ID NO: 51 - protein, 449 aa, Artificial Sequence
  Modified human antibody chain
  THIOETH (127)..(127): Xaa at position 127 is cysteine residue modified by thioether bond formation to maleimide-PEG linker
  THIOETH (381)..(381): Xaa at position 381 is cysteine residue modified by thioether bond formation to maleimide-PEG linker
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPXVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKIKDTLMISRTPEVTCVVVDVSHEDPEVKFNWYVDGVEVHNAKTKPREEQYNSTYRVVSVLTVLHQDWLNGKEYKCKVSNKALPAPEEKTISKQKGQPREPQVYTLPPSRDELTKNQVSLTCLVKGFYPSDIXVEWESNGQPENNYKTTPPVLDSDGSFFLYSKLTVDKSRWQQGNVFSCSVMHEALHNHYTQKSLSLSPG

SEQ ID NO: 52 - protein, 329 aa, Artificial Sequence
  Modified human antibody chain
  THIOETH (40)..(40): Xaa at position 40 is cysteine residue modified by thioether bond formation to maleimide-PEG linker
ASTKGPSVFPLAPSSKSTSGGTAALGCLVKDYFPEPVTVXWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 329 aa, Artificial Sequence
  Modified human antibody chain
  THIOETH (7)..(7): Xaa at position 7 is cysteine residue modified by thioether bond formation to maleimide-PEG linker
  THIOETH (40)..(40): Xaa at position 40 is cysteine residue modified by thioether bond formation to maleimide-PEG linker
ASTKGPXVFPLAPSSKSTSGGTAALGCLVKDYFPEPVTVXWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 326 aa, Artificial Sequence
  Modified human antibody chain
  THIOETH (40)..(40): Xaa at position 40 is cysteine residue modified by thioether bond formation to maleimide-PEG linker
ASTKGPSVFPLAPCSRSTSESTAALGCLVKDYFPEPVTVXWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 55 - protein, 326 aa, Artificial Sequence
  Modified human antibody chain
  THIOETH (45)..(45): Xaa at position 45 is cysteine residue modified by thioether bond formation to maleimide-PEG linker
ASTKGPSVFPLAPCSRSTSESTAALGCLVKDYFPEPVTVSWNSGXLTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 56 - protein, 326 aa, Artificial Sequence
  Modified human antibody chain
  THIOETH (7)..(7): Xaa at position 7 is cysteine residue modified by thioether bond formation to maleimide-PEG linker
  THIOETH (40)..(40): Xaa at position 40 is cysteine residue modified by thioether bond formation to maleimide-PEG linker
  THIOETH (258)..(258): Xaa at position 258 is cysteine residue modified by thioether bond formation to maleimide-PEG linker
ASTKGPXVFPLAPCSRSTSESTAALGCLVKDYFPEPVTVXWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIXVEWESNGQPENNYKTTPPVLDSDGSFFLYSRLTVDKSRWQEGNVFSCSVMHEALHNHYTQKSLSLSLG

SEQ ID NO: 57 - protein, 326 aa, Artificial Sequence
  Modified human antibody chain
  THIOETH (7)..(7): Xaa at position 7 is cysteine residue modified by thioether bond formation to maleimide-PEG linker
  THIOETH (45)..(45): Xaa at position 45 is cysteine residue modified by thioether bond formation to maleimide-PEG linker
  THIOETH (258)..(258): Xaa at position 258 is cysteine residue modified by thioether bond formation to maleimide-PEG linker
ASTKGPXVFPLAPCSRSTSESTAALGCLVKDYFPEPVTVSWNSGXLTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIXVEWESNGQPENNYKTTPPVLDSDGSFFLYSRLTVDKSRWQEGNVFSCSVMHEALHNHYTQKSLSLSLG

SEQ ID NO: 58 - protein, 212 aa, Homo sapiens
RIQMTQSPSSLSASVGDRVTITCKASQDVSIGVAWYQDKPGKAPKLLIYSASYRYTGVPSRFSGSGSGTDFTLTISSLQPEDFATYYCQQYYIYPYTFGQGTKVEIKGQPKAAPSVTLFPPSSEELQANKATLVCYISDFYPGAVTVAWKADSSPVKAGVETTTPSKQSNNKYAAWSYLSLTPEQWKSHRSYSCQVTHEGSTVEKTVAPTEC

SEQ ID NO: 59 - protein, 213 aa, Homo sapiens
DIQMTQSPSSLSASVGDRVTITCSASSSVTYMYWYQRKPGKAPKLLIYDTSNLASGVPSRFSGSGSGTDYTFTISSLQPEDIATYYCQQWSSHIFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0285787 A1
(43) Pub. Date: Aug. 29, 2024

(54) PEPTIDES COMPRISING N-FORMYL-HALOGENATED METHIONINE RESIDUES AND ENGINEERED ANTIBODY-PEPTIDE CONJUGATES THEREOF

(71) Applicant: Eli Lilly and Company, Indianapolis, IN (US)

(72) Inventors: Seamus Patrick BRENNAN, Indianapolis, IN (US); Matthew David LINNIK, Indianapolis, IN (US); Francisco Alcides VALENZUELA, Indianapolis, IN (US)

(21) Appl. No.: 18/567,128

(22) PCT Filed: Jun. 7, 2022
(86) PCT No.: PCT/US2022/032545

§ 371 Date: Dec. 5, 2023

Related U.S. Application Data
(60) Provisional application No. 63/209,762, filed on Jun. 11, 2021.
(60) Provisional application No. 63/210,292, filed on Jun. 14, 2021.

(51) Int. Cl. A61K 47/68; A61P 37/04

(52) U.S. Cl. CPC A61K 47/6811; A61K 47/6855; A61K 47/6889; A61P 37/04

(57) ABSTRACT

Peptides are provided that comprise an N-formyl methionine in which the methyl group of the side chain of methionine has been substituted with one or more halogens such as fluorine. The N-formyl. halogen-substituted methionine exhibits resistance to oxidation. Peptides comprising the N-formyl. halogen- substituted methionine may be utilized as agonists for formyl peptide receptor-1 (FPR-1) and may be conjugated to antibodies or antigen-binding fragments thereof. The antibody conjugates thusly prepared may be utilized to target cells and attract and activate immune cells that comprise the FPR-1 against the targeted cells.

### CROSS REFERENCE TO RELATED APPLICATIONS

The present application claims the benefit of priority to U.S. Provisional Patent Application No. 63/209,762, filed on Jun. 11, 2021, and U.S. Provisional Patent Application No. 63/210,292, filed on Jun. 14, 2021, the entire disclosures of which are incorporated by reference herein.

### SEQUENCE LISTING

A Sequence Listing accompanies this application and is submitted as an ASCII text file of the sequence listing named “083389_01624_ST25.txt” which is 162,084 bytes in size and was created on Jun. 7, 2022. The sequence listing is electronically submitted via EFS-Web with the application and is incorporated herein by reference in its entirety.

### BACKGROUND

The field of the invention relates to peptides comprising N-formyl-halogenated methionine residues and engineered antibodies conjugates comprising the peptides. The peptides and antibody conjugates may be utilized in methods for treating diseases and disorders such as cell proliferative diseases and disorders.

Antibodies and antigen-binding fragments thereof may be conjugated with a variety of payload molecules including therapeutic, cytotoxic, and diagnostic peptides or other small molecules, for in vivo and in vitro applications. In particular, antibody conjugates may be synthesized using native or engineered free cysteine sulfhydryl groups, generated on the surface of immunoglobulin heavy chain or light chain residues, as reactive nucleophiles to form stable chemical linkages with payload molecules, either directly or via a variety of linkers via thiol-conjugation. Antibodies and antigen-binding fragments thereof for conjugation to payload molecules are known in the art.

Antibodies thusly engineered to include a payload molecule may be particularly utilized in cancer immunotherapy. Cancer immunotherapy harnesses the body's immune system to attack cancer cells and is a dynamic area in oncology drug discovery and development. As such, cancer immunotherapy represents a paradigm shift in which the host's immune system is engaged to recognize and destroy tumor cells, in contrast to therapies based on the use of tumoricidal agents (e.g., targeted tumoricidal agents), which may exhibit off-target toxicity. Two successful cancer immunotherapy strategies are: (1) inhibiting suppression of the immune system to enable activation of adaptive and/or innate immune system, especially tumor-directed cytotoxic T-cells (i.e., immune checkpoint blockade), and (2) antibody modifications designed to engage and/or enhance antibody-dependent cell-mediated cytotoxicity (ADCC).

Successful clinical outcomes have recently been achieved with immune checkpoint modulators designed to modify interactions between T-cell surface receptors, such as PD-1 and CTLA-4, and cognate ligand in a manner that results in activation of the T-cells and resulting in T-cell mediated tumor cell destruction. Cancer immunotherapies targeting PD-1 (e.g., nivolumab (Opdivo®) and pembrolizumab (Keytruda®)) and CTLA-4 (e.g., Ipilimumab (Yervoy®) have been FDA approved for the treatment of cancers such as squamous non-small cell lung cancer and metastatic melanoma.

ADCC involves interactions of antibody Fc domains of targeting antibodies with receptors (e.g., Fc gamma receptor IIIa) located on the surface of immune system cells (e.g., natural killer or “NK” cells) resulting in the release of cytolytic proteins from the immune cell with subsequent destruction of the targeted tumor cell. Approved antibody therapies displaying ADCC include Rituxan® (rituximab), Arzerra® (ofatumumab), Herceptin® (trastuzumab) and Campath® (alemtuzumab). Efforts to engineer antibodies with improved ADCC activity via enhanced Fc receptor binding have been effective in patients where antibodies with similar target specificity and less ADCC activation are ineffective or no longer adequately effective in the disease (e.g., Gazyva® obinutuzumab)).

Notwithstanding progress in current cancer immunotherapies, there remains a need for alternative approaches to engage the immune system in treating cancer. For example, the percentage of patients that respond to T-cell directed immunotherapies varies and there is a lack of reliable prognostic assays that identify which patients will respond. In addition, therapy-induced autoimmune disease is a serious side effect associated with immune checkpoint inhibitor therapy. The emergence of autoimmune disease with immune checkpoint inhibitors is likely related to their mechanism of action as they are designed to remove suppression of the T-cell repertoire so that tumor-specific T-cells can emerge, proliferate, and be activated. Thus, they are relatively non-specific, and one consequence of this lack of specificity is that it allows self-reactive T-cells to break tolerance and induce autoimmune disease which is not necessarily reversible on cessation of therapy. Enhanced ADCC approaches are designed to engage the NK cells for tumor cell killing. However, NK cells only constitute about 5% of the total leukocyte population in blood.

Targeting polymorphonuclear cells (PMNs) of the innate immune system to engage in tumor cell killing represents an alternative approach to cancer immunotherapy. PMNs comprise more than 50% of the total leukocyte population, and are a major line of defense against pathogens, including commensal and foreign bacteria. During the innate immune response, pathogen-associated molecular patterns (PAMPs) presented by the pathogen are recognized by pattern recognition receptors (PRRs) located on the surface of immune cells such as neutrophils. One such PRR is formyl peptide receptor 1 (FPR-1), a membrane bound G-protein coupled receptor expressed on the neutrophil cell surface. FPR-1 detects proteins and peptides with N-formyl-methionines including those produced and released by bacteria following infection. Engagement of FPR-1 on the surface of neutrophils with N-formyl-Methionine-containing peptides triggers motility/chemotaxis of neutrophils toward the site of infection. Activation of FPR-1 by formyl peptides also elicits pathogen killing mechanisms such as degranulation in which cytotoxic molecules are released, production of reactive oxygen species (ROS), and phagocytosis in order to destroy the pathogen. There are extensive descriptions of natural and non-natural FPR-I agonists in the literature. (See, e.g., He H Q and Ye R D, Molecules. 2017 Mar. 13; 22(3). pii: E455. doi: 10.3390/molecules22030455; Hwang T L et al., Org Biomol Chem. 2013 Jun. 14; 1 1 (22): 3742-55. doi: 10.1039/c3ob40215k; Cavicchioni G et al., Bioorg Chem. 2006 October; 34(5):298-318; Higgins J D et al., J Med Chem. 1996 Mar. 1; 39(5): 1013-5; Vergelli C et al., Drug Dev Res. 2017 Feb; 78(1):49-62. doi: 10.1002/ddr.21370; Kirpotina L N et al., Mol Pharmacol. 2010 February; 77(2): 159-70. doi: 10.1124/mol. 109.060673; Cilibrizzi A et al., J Med Chem. 2009 Aug. 27; 52(16):5044-57. doi: 10.1021/jm900592h.)

Tumor-targeting therapeutic antibodies capable of engaging PMN neutrophil cells of the innate immune system to participate in tumor cell destruction may also provide advantages over current cancer immunotherapies. For example, such a therapeutic antibody could enhance the T-cell response to the tumor and may not require the presence of tumor-specific T-cells to drive tumor cell killing. Engagement of anti-tumor activity by PMN neutrophils would depend on the presence of FPRs (e.g., FPR-1) which all patients would natively express on neutrophils. Further, an agent that is capable of engaging PMN neutrophils in tumor cell killing would benefit from a robust, continuous supply of tumor killing cells as it has been estimated that 1×10¹¹ neutrophils are produced per day. A tumor targeted antibody capable of engaging neutrophils in tumor cell killing may have safety advantages over immune checkpoint modulators. Unlike checkpoint modulators, neutrophil targeted therapies would not induce or require proliferation of immune cells, as circulating neutrophils are short-lived. In addition, the tumor-targeted antibody is eliminated when neutrophils kill the target tumor cell with the attached antibody, providing a negative feedback loop that diminishes immune stimulation as the therapeutic antibody is consumed by the target effector cells.

Another way that tumor-targeting therapeutic antibodies capable of engaging FPR-1 positive innate immune cells in tumor cell may prove useful is for treatment of cold tumors that have low mutational burden and therefore are not readily recognized by the immune system. Attracting and activating neutrophil-mediated tumor cell killing can result in local production of neoantigens in a cytokine rich environment such that cells of the adaptive immune system acquire the ability to recognize the tumor and target it for elimination.

A tumor targeted antibody capable of engaging neutrophils in tumor cell killing may also have advantages over toxic agent-based antibody drug conjugates (ADC) which are typically designed to release a toxic payload following internalization into the tumor cell. Like ADCs, a tumor targeted antibody capable of engaging neutrophils in tumor cell killing should recognize an antigen with high expression on tumor cells, with low expression on normal tissue, However, unlike ADCs, a tumor targeted antibody capable of engaging neutrophils in tumor cell killing requires agonist exposure to receptors on the surface of innate immune system, and thus is anticipated to function better with target antigens that have relatively less internalization potential.

Antibodies conjugated to n-formyl peptides are disclosed in the art and may be referred to as “bactabodies” based on the occurrence of n-formyl peptides in bacteria. (See, e.g., WO2018/232088, the content of which is incorporated herein by reference in its entirety). One difficulty in utilizing antibodies conjugated to N-formyl-methionine containing peptides as targeting agents and agonists for attracting and activating cells comprising FPR-1 receptors is that N-formyl-methionines are subject to oxidation of the sulfur atom and formation of methyl-sulfoxide or generation of Met(O) in vivo. The oxidation of the sulfur atom of the methionine residue results in a significant reduction in efficacy of the N-formyl methionine as an agonist for FPR-1. Therefore, N-formyl-methionines and peptides containing N-formyl methionines that are resistant to oxidation and that function as agonists for FPR-1 are desirable.

### SUMMARY

Disclosed are peptides that comprise an N-formyl methionine in which the methyl group of the side chain of methionine may be substituted with one or more halogens such as fluorine. The N-formyl, halogen-substituted methionine exhibits resistance to oxidation. Peptides comprising the N-formyl, halogen-substituted methionine may be utilized as agonists for formyl peptide receptor (FPR) and may be conjugated to antibodies or antigen-binding fragments thereof. The antibody conjugates thusly prepared may be utilized to target cells and attract and activate immune cells that comprise the FPR against the targeted cells.

### BRIEF DESCRIPTION OF THE FIGURES

FIG. 1. Oxidation of N-formyl methionine to N-formyl methionine S-oxide. N-formyl methionine (CF₃) is resistant to S-oxidation.

FIG. 2. Exemplary synthesized peptides-linkers. frm: formyl; MIFL: Met-Ile-Phe-Leu; Peg: polyethylene glycol monomer; M(CF₃): trifluoromethyl methionine; Dpg: di-n-propylglycine; 2Nal: 2-naphthylalanine; αMeF: alpha-methyl-phenylalanine; Nle: nor-leucine; γE: glutamic acid residue connected through its side-chain gamma carboxyl group; εK: lysine residue connected through its side-chain epsilon amino group.

FIG. 3. Exemplary synthesized peptides-linkers. frm: formyl; MIFL: Met-Ile-Phe-Leu; Peg: polyethylene glycol monomer; Mal: maleimide; MLF: Met-Leu-Phe; Dpg: di-n-propylglycine; 2Nal: 2-naphthylalanine; αMeF: alpha-methyl-phenylalanine; Nle: nor-leucine; D-Nle: D-nor-leucine; γE: glutamic acid residue connected through its side-chain gamma carboxyl group; M(O): oxidized methionine (control); M(CF₃): trifluoromethyl methionine. Provided formula is for FRM-046 (FRM-047 w/o Mal).

FIG. 4. Exemplary synthesized peptides-linkers. frm: formyl; MIFL: Met-Ile-Phe-Leu; Peg: polyethylene glycol monomer; Mal: maleimide; MLF: Met-Leu-Phe; Dpg: di-n-propylglycine; 2Nal: 2-naphthylalanine; αMeF: alpha-methyl-phenylalanine; Nle: nor-leucine; D-Nle: D-nor-leucine; γE: glutamic acid residue connected through its side-chain gamma carboxyl group; M(O): oxidized methionine (control); M(CF₃): trifluoromethyl methionine; 4-Pal: 4-pyridyl-alanine.

FIG. 5. Chemistry for preparing Fmoc-L-trifluoromethionine from Fmoc-S-trityl-L-homocysteine.

FIG. 6. Reactive oxygen species (ROS) production in neutrophils activated with various peptides.

FIG. 7. Reactive oxygen species (ROS) production in neutrophils activated with various peptides.

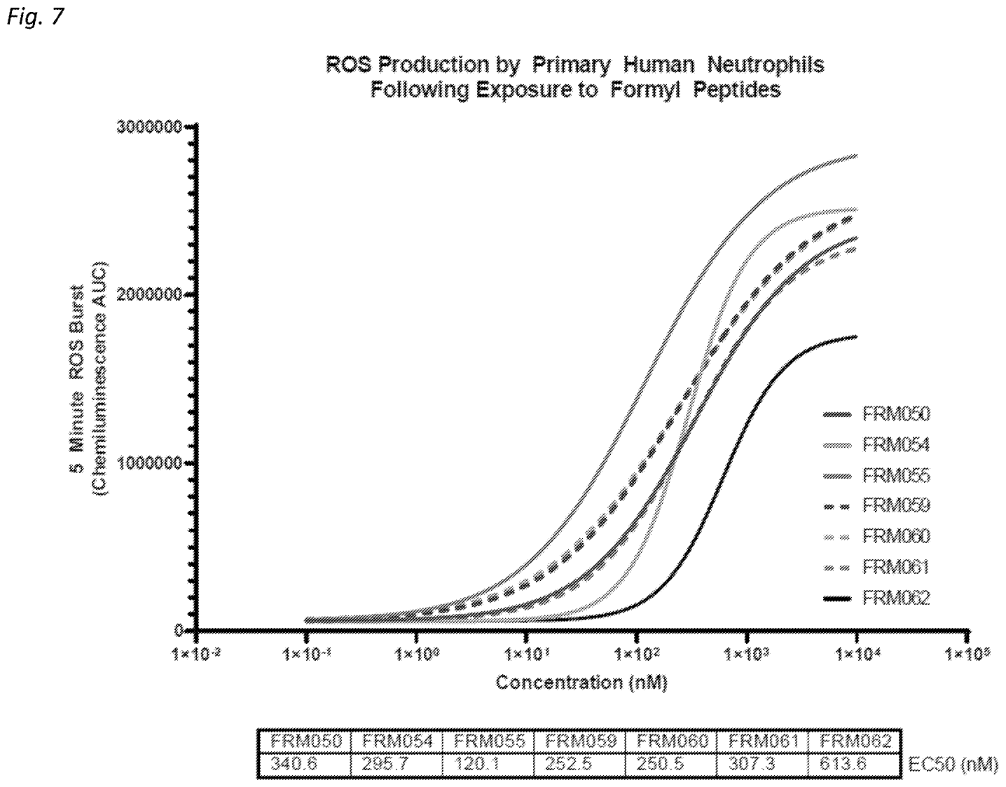

FIG. 8. Neutrophil chemotaxis following exposure to peptides.

FIG. 9. Reactive oxygen species (ROS) production in neutrophils activated with peptide conjugated to trastuzumab.

FIG. 10A. Pharmacokinetics for trastuzumab.

FIG. 10B. Pharmacokinetics for trastuzumab conjugated to peptide FRM047.

FIG. 10C. Tabular data for results in FIG. 9A and FIG. 9B.

FIG. 10D. PK Parameters Using 2-Compartment Model for results in FIG. 9A and FIG. 9B.

FIG. 11. Exposure Profile of Trastuzumab Parent Antibody and Trastuzumab Bactabody with frm-Met(CF₃) FRM-058 shows similar exposure between Tmab bactabody compared to Tmab parent.

### DETAILED DESCRIPTION

The present invention is described herein using several definitions, as set forth below and throughout the application.

Unless otherwise specified or indicated by context, the terms “a”, “an”, and “the” mean “one or more.” For example, “a peptide,” “a linker,” and “an antibody,” should be interpreted to mean “one or more peptides,” “one or more linkers,” and “one or more antibodies,” respectively.

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus≤10% of the particular term and “substantially” and “significantly” will mean plus or minus>10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, “a subject in need thereof,” refers to a human or non-human mammal, and more preferably a human, which has been diagnosed as having a condition or disorder for which treatment or administration with the peptides and conjugates disclosed herein is indicated.

As used herein, “a subject in need thereof” may include a subject having or at risk for developing a disease or disorder that may be treated and/or prevented by modulating an immune response in the subject. As disclosed herein, “modulation” may include induction and/or enhancement of an immune response in a subject.

A subject in need thereof may include a subject having or at risk for developing a cell proliferative disease or disorder. Cell proliferative diseases and disorders may include but are not limited to cancer such as breast cancer, lung cancer, prostate cancer, skin cancer, colorectal cancer, bladder cancer, kidney cancer, liver cancer, thyroid cancer, endometrial cancer, muscle cancer, bone cancer, mesothelial cancer, vascular cancer, fibrous cancer, leukemia or lymphoma.

As used herein the term “effective amount” refers to the amount or dose of a conjugated antibody compound of the present invention, which upon single or multiple dose administration to the patient, provides the desired pharmacological effect in the patient. An effective amount can be readily determined by the attending diagnostician, as one skilled in the art, by considering a number of factors such as the species of mammal; its size, age, and general health; the specific disease or surgical procedure involved, the degree or severity of the disease or malady; the response of the individual patient; the particular compound or composition administered; the mode of administration; the bioavailability characteristics of the preparation administered; the dose regimen selected; and the use of any concomitant medications.

The disclosed subject matter relates to peptides and polypeptides which may include fusion polypeptides and conjugates. As used herein, the terms “peptide” or “polypeptide” or “protein” may be used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” typically is defined as a short polymer of amino acids, of a length typically of 50, 40, 30, 20 or less amino acids.

A “polypeptide,” “protein,” or “peptide” as contemplated herein typically comprises a polymer of proteinogenic amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) or non-proteinogenic amino acids as contemplated herein.

The term “proteinogenic amino acids” refers to those amino acids that are found in naturally occurring proteins and may be referred to as “coding amino acids.” The term “non-proteinogenic amino acids” refers to those amino acids that are not found in naturally occurring proteins and may be referred to as “non-coding amino acids.” As such, the term “non-proteinogenic amino acid” means an amino acid other than alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

The word “fusion” refers to a polypeptide sequence comprising an exogenous amino acid sequence fused to a native amino acid sequence. The exogenous sequence may be fused at the N-terminus of the native amino acid sequence, at the C-terminus of the native amino acid sequence, or internally within the native amino acid sequence such that the fusion protein comprising an N-terminal portion of the native amino acid sequence, the exogenous amino acid sequence, and a C-terminal portion of the native amino acid sequence.

The word “conjugate” refers to a molecule in which two components which are not natively covalently bound are covalently bound, either directly or via a linking group. A conjugate may include a peptide or polypeptide which has been covalently bounded to an antibody or an antigen-binding fragment thereof. The disclosed conjugates may be covalently bonded via a bond formed between reactive group present on a peptide or polypeptide and a reactive group present on an antibody or an antigen-binding fragment thereof. In some embodiments, the bond may be formed between an electrophilic reactive group present on a peptide or polypeptide and a nucleophilic reactive group present on an antibody or an antigen-binding fragment thereof. Electrophilic reactive groups may include, but are not limited to, maleimide groups, maleimide-diaminopropionate groups, iodoacetamide groups, or vinyl sulfone groups. Nucleophilic reactive groups may include, but are not limited to, free thiol groups (i.e., reduced di-thio bonds).

The disclosed subject matter relates to antibodies and antigen-binding fragments thereof. Unless indicated otherwise, the term “antibody” refers to an immunoglobulin molecule comprising two heavy chains and two light chains interconnected by disulfide bonds. The amino terminal portion of each chain includes a variable region of about 100 to about 110 amino acids primarily responsible for antigen recognition via the complementarity determining regions (CDRs) contained therein. The carboxy-terminal portion of each chain defines a constant region primarily responsible for effector function.

As used herein, the term “antigen-binding fragment” refers to any antibody fragment that retains the ability to bind to its antigen. Such “antigen-binding fragments” may include but are not limited to Fv, scFv, Fab, F(ab′)₂, Fab′, scFv-Fc fragments, and diabodies. An antigen-binding fragment of an antibody will typically comprise at least one variable region. Preferably, an antigen-binding fragment comprises a heavy chain variable region (HCVR) and a light chain variable region (LCVR). More preferably, an antigen-binding fragment as used herein comprises a HCVR and a LCVR which confers antigen-binding specificity to an epitope of a targeted antigen.

As used herein, the term “light chain variable region (LCVR)” refers to a portion of a LC of an antibody molecule that includes amino acid sequences of Complementarity Determining Regions (CDRs; i.e., LCDR1, LCDR2, and LCDR3), and Framework Regions (FRs).

As used herein, the term “heavy chain variable region (HCVR)” refers to a portion of a HC of an antibody molecule that includes amino acid sequences of Complementarity Determining Regions (CDRs; i.e., HCDR1, HCDR2, and HCDR3), and Framework Regions (FRs).

As used herein, the terms “complementarity determining region” and “CDR”, refer to the non-contiguous antigen combining sites found within the variable region of LC and HC polypeptides of an antibody or an antigen-binding fragment thereof.

The CDRs are interspersed with regions that are more conserved, termed framework regions (“FR”). Each LCVR and HCVR is composed of three CDRs and four FRs, arranged from amino-terminus to carboxy-terminus in the following order: PRE CDR1, FR2, CDR2, FR3, CDR3, FR4. The three CDRs of the light chain are referred to as “LCDR1, LCDR2, and LCDR3” and the three CDRs of the HC are referred to as “HCDR1, HCDR2, and HCDR3.” The CDRs contain most of the residues which form specific interactions with the antigen. The numbering and positioning of CDR amino acid residues within the LCVR and HCVR regions is in accordance with known conventions.

Commonly used numbering conventions include the “Kabat Numbering” and “EU Index Numbering” systems. “Kabat Numbering” or “Kabat Numbering system”, as used herein, refers to the numbering system devised and set forth by the authors in Kabat et al., Sequences of Proteins of Immunological Interest, 5th Ed, Public Health Service, National Institutes of Health, Bethesda, MD (1991) for designating amino acid residues in both variable and constant domains of antibody heavy chains and light chains. “EU Index Numbering” or “EU Index Numbering system”, as used herein, refers to the numbering convention for designating amino acid residues in antibody heavy chain constant domains, and is also set forth in Kabat et al (1991). Other conventions that include corrections or alternate numbering systems for variable domains include Chothia (Chothia C, Lesk A M (1987), J Mol Biol 196: 901-917; Chothia, et al. (1989), Nature 342: 877-883), IMGT (Lefranc, et al. (2003), Dev Comp Immunol 27: 55-77), and AHo (Honegger A, Pluckthun A (2001) J Mol Biol 309: 657-670). Unless otherwise expressly stated herein, all references to immunoglobulin heavy chain constant region C|−|1, hinge, C|−|2, and C|−|3 amino acid residues (i.e., numbers) appearing in the specification, Examples and Claims are based on the EU Index Numbering.

The general structure of an “IgG antibody” is very well-known. A wild type (WT) antibody of the IgG type is hetero-tetramer of four polypeptide chains (two identical heavy chains and two identical light chains) that are cross-linked via intra- and interchain disulfide bonds. Each heavy chain (HC) is comprised of an N-terminal heavy chain variable region (“V_{H}”) and a heavy chain constant region (“CH”). The heavy chain constant region is comprised of three domains (C_{H}1, C_{H}2, and C_{H}3) as well as a hinge region (“hinge”) between the C_{H}1 and C_{H}2 domains. Each light chain (LC) is comprised of an N-terminal light chain variable region (“V_{L}”) and a light chain constant region (“C_{L}”). The V_{L} and C_{L} regions may be of the kappa (“κ”) or lambda (“λ”) isotypes (“C_{κ}” or “C_{λ}”, respectively). Each heavy chain associates with one light chain via interfaces between the heavy chain and light chain variable domains (the V_{H}/V_{L} interface) and the heavy chain constant C_{H}1 and light chain constant domains (the C_{H}1/C_{L} interface). The association between each of the V_{H}-C_{H}1 and V_{L}-C_{L} segments forms two identical antigen binding fragments (Fabs) which direct antibody binding to the same antigen target or epitope. Each heavy chain associates with the other heavy chain via interfaces between the hinge-C_{H}2-C_{H}3 segments of each heavy chain, with the association between the two C_{H}2-C_{H}3 segments forming the Fc region of the antibody. Together, each Fab and the Fc form the characteristic Y-shaped” architecture of IgG antibodies, with each Fab representing the “arms” of the ‘Y.” IgG antibodies can be further divided into subtypes, e.g., lgG1, lgG2, lgG3, and lgG4 which differ by the length of the hinge regions, the number and location of inter- and intra-chain disulfide bonds and the amino acid sequences of the respective HC constant regions

The variable regions of each heavy chain-light chain pair associate to form binding sites. The heavy chain variable region (V_{H}) and the light chain variable region (V_{L}) can be subdivided into regions of hypervariability, which are the complementarity determining regions (“CDRs”), interspersed with regions that are more conserved, which are the framework regions (“FR”). Each V_{H} and V_{L} is composed of three CDRs and four FRs, arranged from amino-terminus to carboxy-terminus in the following order: FR1, CDR1, FR2, CDR2, FR3, CDR3, FR4. CDRs of the heavy chain may be referred to as “CDRH1, CDRH2, and CDRH3” and the 3 CDRs of the light chain may be referred to as “CDRL1, CDRL2 and CDRL3.” The FRs of the heavy chain may be referred to as HFR1, HFR2, HFR3, and HFR4 whereas the FRs of the light chain may be referred to as LFR1, LFR2, LFR3, and LFR4. The CDRs contain most of the residues which form specific interactions with the antigen.

The antibodies and antigen-binding fragments thereof for use in the disclosed conjugates can be produced using techniques well known in the art, such as recombinant expression in mammalian or yeast cells. In particular, the methods and procedures of the Examples herein may be readily employed. In addition, the antibodies and antigen-binding fragments of the present invention may be further engineered to comprise framework regions derived from fully human frameworks. A variety of different human framework sequences may be used in carrying out embodiments of the present invention. As a particular embodiment, the framework regions employed in the antibodies and antigen-binding fragments of the present conjugates are of human origin or are substantially human (at least 95%, 97% or 99% of human origin.) The sequences of framework regions of human origin are known in the art and may be obtained from The Immunoglobulin Factsbook, by Marie-Paule Lefranc, Gerard Lefranc, Academic Press 2001, ISBN 012441351.

Expression vectors capable of directing expression of genes to which they are operably linked are well known in the art. Expression vectors contain appropriate control sequences such as promoter sequences and replication initiation sites. They may also encode suitable selection markers as well as signal peptides that facilitate secretion of the desired polypeptide product(s) from a host cell. The signal peptide can be an immunoglobulin signal peptide or a heterologous signal peptide. Nucleic acids encoding desired polypeptides, for example the HC and LC components of the conjugated IgG antibodies of the present invention, may be expressed independently using different promoters to which they are operably linked in a single vector or, alternatively, the nucleic acids encoding the desired products may be expressed independently using different promoters to which they are operably linked in separate vectors. Single expression vectors encoding both the HC and LC components of the cysteine-engineered IgG antibodies of the present invention may be prepared using standard methods.

As used herein, a “host cell” refers to a cell that is stably or transiently transfected, transformed, transduced or infected with nucleotide sequences encoding a desired polypeptide product or products. Creation and isolation of host cell lines producing an IgG antibody for use in the present invention can be accomplished using standard techniques known in the art. Mammalian cells are preferred host cells for expression of the cysteine-engineered IgG antibodies according to the present invention. Particular mammalian cells include HEK293, NSO, DG-44, and CHO cells. Preferably, assembled proteins are secreted into the medium in which the host cells are cultured, from which the proteins can be recovered and isolated. Medium into which a protein has been secreted may be purified by conventional techniques. For example, the medium may be applied to and eluted from a Protein A or G column using conventional methods. Soluble aggregate and multimers may be effectively removed by common techniques, including size exclusion, hydrophobic interaction, ion exchange, hydroxyapatite or mixed modal chromatography. Recovered products may be immediately frozen, for example at −70° C., or may be lyophilized. As one of skill in the art will appreciate, when expressed in certain biological systems, e.g., mammalian cell lines, antibodies are glycosylated in the Fc region unless mutations are introduced in the Fc to reduce glycosylation. In addition, antibodies may be glycosylated at other positions as well.

New chemical entities and uses for chemical entities are disclosed herein for example in the form of peptides and conjugates of the disclosed peptides. The chemical entities may be described using terminology known in the art and further discussed below.

The term “alkyl” as contemplated herein includes a straight-chain or branched alkyl radical in all of its isomeric forms, such as a straight or branched group of 1-12, 1-10, or 1-6 carbon atoms, referred to herein as C1-C12 alkyl, C1-C10-alkyl, and C1-C6-alkyl, respectively.

The term “alkylene” refers to a diradical of straight-chain or branched alkyl group (i.e., a diradical of straight-chain or branched C1-C6 alkyl group). Exemplary alkylene groups include, but are not limited to —CH₂—, —CH₂CH₂—, —CH₂CH₂CH₂—, —CH(CH₃)CH₂—, —CH₂CH(CH₃)CH₂—, —CH(CH₂CH₃)CH₂—, and the like.

The term “haloalkyl” refers to an alkyl group that is substituted with at least one halogen. For example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). One type of heteroalkyl group is an “alkoxy” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C2-C12-alkenyl, C2-C10-alkenyl, and C2-C6-alkenyl, respectively.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C2-C12-alkynyl, C2-C10-alkynyl, and C2-C6-alkynyl, respectively.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C4-8-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido or carboxyamido, amidino, amino, aryl, arylalkyl, azido, carbamate, carbonate, carboxy, cyano, cycloalkyl, ester, ether, formyl, halo, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloheteroalkyl” refers to a monovalent saturated cyclic, bicyclic, or bridged cyclic hydrocarbon group of 3-12, 3-8, 4-8, or 4-6 carbons in which at least one carbon of the cycloalkane is replaced with a heteroatom such as, for example, N, O, and/or S.

The term “cycloalkylene” refers to a cycloalkyl group that is unsaturated at one or more ring bonds ..

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number of ring carbon atoms. For example, the partially unsaturated carbocyclyl may contain 5-14, 5-12, 5-8, or 5-6 ring carbon atoms, and accordingly be referred to as a 5-14, 5-12, 5-8, or 5-6 membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido or carboxyamido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido or carboxyamido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C3-C7 heterocyclyl group refers to a saturated or partially unsaturated 3- to 7-membered ring structure containing one to four heteroatoms, such as nitrogen, oxygen, and sulfur. The designation “C3-C7” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines (e.g., mono-substituted amines or di-substituted amines), wherein substituents may include, for example, alkyl, cycloalkyl, heterocyclyl, alkenyl, and aryl.

The terms “alkoxy” or “alkoxyl” are art-recognized and refer to an alkyl group, as defined above, having an oxygen radical attached thereto. Representative alkoxy groups include methoxy, ethoxy, tert-butoxy and the like.

An “ether” is two hydrocarbons covalently linked by an oxygen atom or sulfur atom. Accordingly, the substituent of an alkyl that renders that alkyl an ether is or resembles an alkoxyl or thiol.

The term “carbonyl” as used herein refers to the radical —C(O)—.

The term “oxo” refers to a divalent oxygen atom —O—.

The term “carboxamido” as used herein refers to the radical —C(O)NRR′, where R and R′ may be the same or different. R and R′, for example, may be independently hydrogen, alkyl, aryl, arylalkyl, cycloalkyl, formyl, haloalkyl, heteroaryl, or heterocyclyl.

The term “carboxy” as used herein refers to the radical —COOH or its corresponding salts, e.g. —COONa, etc.

The term “amide” or “amido” or “amidyl” as used herein refers to a radical of the form —R¹C(O)N(R²)—, —R¹C(O)N(R²)R³—, —C(O)NR²R³, or —C(O)NH₂, wherein R¹, R² and R³, for example, are each independently hydrogen, alkyl, alkoxy, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure (e.g., peptides and conjugates thereof) may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” or “+” or “−” depending on the configuration of substituents around the stereogenic carbon atom and or the optical rotation observed. The present invention encompasses various stereo isomers of these compounds and mixtures thereof. Stereoisomers include enantiomers and diastereomers. Mixtures of enantiomers or diastereomers may be designated (±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Also contemplated herein are compositions comprising, consisting essentially of, or consisting of an enantiopure compound, which composition may comprise, consist essential of, or consist of at least about 50%, 60%, 70%, 80%, 90%, 95%, 96%, 97%, 98%, 99%, or 100% of a single enantiomer of a given compound (e.g., at least about 99% of an R enantiomer of a given compound).

The polypeptide chains described herein may be depicted by their sequence of amino acids from N-terminus to C-terminus, when read from left to right, with each amino acid represented by either their single letter or three-letter amino acid abbreviation. Unless otherwise stated herein, all amino acids used in the preparation of the polypeptides of the present invention are L-amino acids, the istereoisomers being D-amino acids. The “N-terminus” (or amino terminus) of an amino acid, or a polypeptide chain, refers to the free amine group on the amino acid, or the free amine group on the first amino acid residue of the polypeptide chain. Further, the term “N-terminal amino acid” refers to the first amino acid in a polypeptide chain. Likewise, the “C-terminus” (or carboxy terminus) of an amino acid, or a polypeptide chain, refers to the free carboxy group on the amino acid, or the free carboxy group on the final amino acid residue of the polypeptide chain. Further, the term “C-terminal amino acid” refers to the last amino acid in a polypeptide chain.

As used herein, the phrase “ . . . a/an [amino acid name] substituted at residue . . . ”, in reference to a heavy chain or light chain polypeptide, refers to substitution of the parental amino acid with the indicated amino acid. By way of example, a heavy chain comprising “an alanine substituted at residue 235” refers to a heavy chain wherein the parental amino acid sequence has been mutated to contain an alanine at residue number 235 in place of the parental amino acid. Such mutations may also be represented by denoting a particular amino acid residue number, preceded by the parental amino acid and followed by the replacement amino acid. For example, “F235A” refers to a replacement of a phenylalanine at residue 235 with an alanine. Similarly, “235A” refers to replacement of a parental amino acid with an alanine. An “engineered” cysteine refers to substitution of the parental amino acid with a cysteine.

As used herein, “N-formyl-methionine peptide” refers to a peptide, wherein the N-terminal amino acid is a formylated methionine. The N-formyl-methionine residues of the disclosed peptides may comprise one or more halogen substitutions. As such, the N-formyl, halogen-substituted methionine residues may have a formula represented as:

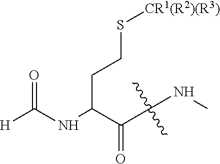

wherein R¹, R², and R³ are independently selected from hydrogen and halogen (e.g., F, Cl, Br, or I), and at least one of R¹, R², and R³, preferably at least two of R¹, R², and R³ are halogen, and more preferably each of R¹, R², and R³ is halogen (e.g., wherein the methionine residue comprises C(halogen); at the terminus of the side chain). As used herein, “N-formyl-CF₃-methionine peptide refers to a peptide, wherein the N-terminal amino acid is a formylated methionine comprising a trifluoro-substituted methyl group at the terminus of the methionine side chain.

As used herein, “linker” refers to a structure that connects two or more additional structures. Examples of linkers include peptide linkers, protein linkers, PEG linkers, and combinations thereof. A “maleimide-PEG linker”, as used herein, refers to a chemical moiety comprising a polyethylene glycol (PEG) polymer of the formula “—(O—CH₂—CH₂)n-” wherein “n” is 3-24, and a derivatized maleimide functional group, wherein said linker may form a covalent attachment to an antibody or an antigen-binding fragment thereof through a thioether bond between a maleimide functional group and a cysteine residue in the antibody or an antigen-binding fragment, and/or or may form a covalent attachment to an N-formyl-methionine peptide through an amide bond to the epsilon amino side chain of the C-terminal lysine of a N-formyl-methionine peptide or an amide bond to the gamma carboxyl group of the C-terminal glutamic acid of a N-formyl-methionine peptide.

As illustrated, the disclosed peptides and conjugates may include one or more polyethylene glycol (PEG) polymer linking sequences, and may be considered to be pegylated. As one of skill in the art will appreciate, pegylation reagents are often described by reference to the molecular weight (in daltons or kilodaltons) of the PEG polymer portion of the PEG-containing compounds in the reagent. Further, many commercially available PEG-containing reagents generally have some degree of polydispersity, meaning that the number of repeating ethylene glycol monomer units contained within the reagent (the “n”) varies over a range, typically over a narrow range. Thus, the reference to the PEG polymer molecular weight in a reagent is typically a reference to the average molecular weight of the PEG polymers contained within the reagent. The ethyl-oxy monomer —(O—CH₂—CH₂)— of the reagent used to prepare the conjugated antibody compounds of the present invention has a molecular weight of about 44 g/mol or 44 daltons. Thus, one of skill in the art can readily determine the value of “n” when using a pegylation reagent denoted by its average molecular weight and, likewise, the value of “n” in a resulting conjugated antibody compound.

As used herein, a formyl group consists of a carbonyl bonded to a hydrogen and is represented by the formula, CH(O)—, or

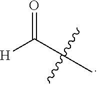 [SMILES: *C=O]

The N-terminal methionine residues may include a formyl group (i.e., an N-formyl substitution on the N-terminal nitrogen atom).

Maleimide refers to the structure:

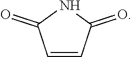

Maleimide-containing moieties which may be utilized to conjugate the molecules disclosed herein may include maleimide-diaminopropionic acid having a structure:

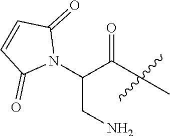 [SMILES: *C(=O)C(CN)N1C(=O)C=CC1=O]

and

maleimidopropionic acid having a structure:

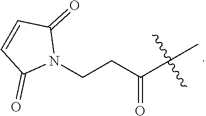 [SMILES: *C(=O)CCN1C(=O)C=CC1=O]

### Peptides Comprising N-Formyl-Halogenated Methionine Residues and Engineered Antibody-Peptide Conjugates Thereof.

The disclosed subject matter relates to peptides that comprise an N-formyl methionine in which the methyl group of the side chain of methionine has be substituted with one or more halogens such as fluorine. The N-formyl, halogen-substituted methionine exhibits resistance to oxidation. Peptides comprising the N-formyl, halogen-substituted methionine may be utilized as agonists for formyl peptide receptor (FPR-1) and may be conjugated to antibodies or antigen-binding fragments thereof. The conjugates thusly prepared may be utilized to target cells and attract and activate immune cells that comprise the FPR-1 against the targeted cells.

In some embodiments, the disclosed subject matter relates to conjugated antibodies or antigen-binding fragments thereof. The conjugates comprise an antibody or an antigen-binding fragment thereof that is conjugated to a peptide comprising an N-formyl, halogen-substituted methionine residue at the N-terminus of the peptide. Suitable N-formyl-halogenated methionine residues may include, but are not limited to N-formyl-trifluorinated methionines. N-formyl, fluorine-substituted methionines may be prepared using methods disclosed in the art. (See, e.g., Houston et al., Biorg & Medic. Chem. Lett,” Vol. 7, No. 23, pp. 3007-3012, 1997, the content of which is incorporated herein by reference in its entirety).

The peptide and the antibody or antigen-binding fragment thereof may be conjugated directly via a reactive group in the peptide and reactive group in the antibody or antigen-binding fragment. Alternative, the peptide and the antibody or antigen-binding fragment thereof may be conjugated via a linker having a reactive group for conjugating the peptide and having a reactive group for conjugating the antibody or antigen-binding fragment thereof. In some embodiments, the conjugate may have a formula represented as:

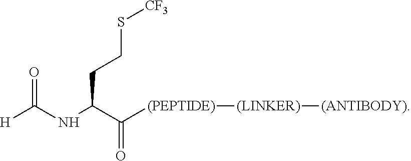

The components of the disclosed conjugates, i.e., the peptide, the optional linker, and the antibody or antigen-binding fragment thereof may be conjugated via bonds formed between any suitable reactive groups. In some embodiments, the peptide comprises a C-terminal glutamic acid residue and the peptide is conjugated to the linker via an amide bond formed between the gamma carboxyl group of the glutamic acid and an amino group of the linker. In other embodiments, the peptide comprises a C-terminal lysine residue and the peptide is conjugated to the linker via an amide bond formed between the epsilon amino group of the lysine and a carboxyl group of the linker.

The disclosed conjugates may comprise multiple peptides, multiple linkers, and/or multiple antibodies or antigen-binding fragments thereof. In some embodiments, the conjugates comprise at least two peptides and linkers and may form a branched structure. In some embodiments, the conjugates have a formula represented as:

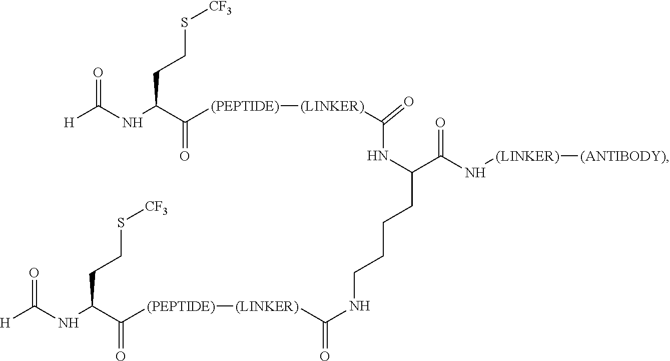

which may be characterized as a branched structure.

The peptides disclosed herein typically include an N-terminal, N-formyl, halogen-substituted methionine residue. The peptides typically comprise additional amino acids and in some embodiments, the peptides may comprise 2-50 amino acids (or 2-40, 2-30, 2-20, or 2-10 amino acids) amino acids bonded via peptide bonds formed between amino groups and carboxyl groups in the backbone or side-chains of the amino acids. Preferably, the disclosed peptides are resistant to cleavage by endopeptidases, for example, endopeptidases that are associated with neutrophils and integral membrane endopeptidases in particular. In some embodiments, the disclosed peptides are resistant to cleavage by endopeptidase 24.11 (EP 24.11; E.C.3.4.24.11, also called enkephalinase neutral endopeptidase, CALLA, CD10, or neprilysin); and/or endopeptidase 24.15 (EP 24.15; E.C.3.4.24.15) a metallopeptidase found within alveolar macrophages, monocytes, T lymphocytes, and B lymphocytes; and/or CD13/aminopeptidase N (CD13/APN); and/or BP-1/6C3/aminopeptidase A (BP-1/6C3/APA); and CD26/dipeptidyl peptidase IV (CD26/DPPIV).

In some embodiments, the disclosed peptides and conjugates thereof comprise one or more non-proteinogenic amino acids including N-formyl, halogen-substituted methionine at the N-terminus and optionally one or more non-proteinogenic amino acids other than N-formyl, halogen-substituted methionine. Preferably, the non-proteinogenic amino acids and/or the bonds formed between the non-proteinogenic amino acids render the peptides resistant to cleavage by endopeptidases as disclosed herein.

As would be understood in the art, non-proteinogenic amino acids are amino acids that are not coding amino acids in an organism and are not observed to be naturally present in proteins. Proteinogenic amino acids include L-amino acid forms of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. Therefore, non-proteinogenic amino acids may be defined as an amino acid (i.e., a molecule comprising a free amino group and a free carboxyl group bonded to an α-carbon atom) which is not any of L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, and L-valine. For example, a non-proteinogenic amino acid may have a formula, NH₂—C(R)—COOH, wherein R is not a side chain of any of the coding, proteinogenic amino acids.

In some embodiments, the disclosed peptides and conjugates thereof comprise one or more non-proteinogenic amino acids selected from D-amino acids. Suitable D-amino acids may include, but are not limited to D-amino acids of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and/or valine.

In some embodiments, the disclosed peptides comprise non-proteinogenic amino acids which are homologues of coding amino acids that lack one or more methylene groups (—CH₂—) between the a-carbon and the side chain of the amino acid. Suitable homologues for use as non-proteinogenic amino acids of the disclosed peptides and conjugates thereof may include, but are not limited to 2-aminoisobutyric acid, 2-amino-2-hydroxyacetic acid, 2α-methyl-2-hydroxy-glycine, 2-amino-2-methylbutyric acid (i.e., isovaline), methylcysteine, azetidine-2-carboxylic acid, phenylglycine, 4-hydroxyphenylglycine, 3-indolylglycine, aminomalonic acid, 2,3-diamino-3-oxopropanoic acid, 2-amino-2-(1H-imidazol-5-yl)acetic acid, ornithine, 2,4-diaminobutanoic acid, 2,3-diaminopropionic acid, and 2-amino-4-(diaminomethylideneamino)butanoic acid.

In some embodiments, the disclosed peptides comprise non-proteinogenic amino acids which are homologues of coding amino acids which possess one or more additional methylene groups (—CH₂—) between the a-carbon and the side chain (e.g., homo-amino acids possessing a single additional methylene group (—CH₂—), bishomo-amino acids possessing two additional methylene groups (—CH₂—CH₂—), and the like). Suitable homologues for use as non-proteinogenic amino acids of the disclosed peptides and conjugates thereof may include, but are not limited to homo-amino acids of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine, such as homo-alanine, homo-arginine, homo-glutamine, homo-glutamic acid, homo-isoleucine, homo-leucine, homo-lysine, homo-methionine, homo-phenylalanine, homo-proline (i.e., piperidine-2-carboxylic acid), homo-serine, homo-threonine, homo-tryptophan, and homo-tyrosine. Suitable homologues for use as non-proteinogenic amino acids of the disclosed peptides and conjugates thereof may include, but are not limited to bishomo-amino acids of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine.

In some embodiments, the disclosed peptides comprise non-proteinogenic amino acids which are alkylated amino acids comprising an alkyl substitution (e.g., a C₁-C₆ alkyl substitution such as methyl) on the α-carbon. Suitable alkyl-substituted amino acids may include α-carbon, alkyl substituted alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine, such as 2-methyl-serine (i.e., α-methyl-serine), 2-methyl-threonine (i.e., α-methyl-threonine), α-methyl-valine, α-methyl-leucine, 2-amino-2,3-dimethyl-pentanoic acid (i.e., α-methyl-isoleucine), α-methyl-methionine, α-methyl-cysteine, 2-methyl-proline, α-methyl-phenylalanine, α-methyl-tyrosine, α-methyl-tryptophan, 2-methyl-aspartic acid, 2-methyl-glutamic acid, 2,4-diamino-2-methyl-4-oxobutanoic acid (i.e., α-methyl-asparagine), 2,5-diamino-2-methyl-5-oxopentanoic acid (i.e., α-methyl-glutamine), α-methyl-histidine, α-methyl-lysine, and 2-methyl-arginine (i.e., α-methyl-lysine). In some embodiments, the disclosed peptides comprise non-proteinogenic amino acids which are di-alkylated amino acids comprising a di-alkyl substitution (e.g., a C₁-C₆ di-alkyl substitution such as di-methyl) on the α-carbon. Suitable di-alkylated substituted amino acids may comprise α-carbon, di-alkyl substituted glycine, such as di-n-propylglycine (Dpg).

In some embodiments, the disclosed peptides comprise non-proteinogenic amino acids which are alkylated amino acids comprising an alkyl substitution (e.g., a C₁-C₆ alkyl substitution such as methyl) on the amino group. Suitable N-alkylated amino acids may include N-alkylated alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine, (such as N-methyl-alanine, N-methyl-arginine, N-methyl-asparagine, N-methyl-aspartic acid, N-methyl-cysteine, N-methyl-glutamic acid, N-methyl-glutamine, N-methyl-glycine, N-methyl-histidine, N-methyl-isoleucine, N-methyl-leucine, N-methyl-lysine, N-methyl-methionine, N-methyl-phenylalanine, N-methyl-proline, N-methyl-serine, N-methyl-threonine, N-methyl-tryptophan, N-methyl-tyrosine, and N-methyl-valine).

In some embodiments, the disclosed peptides comprise non-proteinogenic amino acids selected from phenylalanine, tyrosine, tryptophan, histidine, proline, naphthylalanine, which optionally include a ring substitution selected from C₁-C₆ alkyl substitutions, halogen-substitutions, and cyano-substitutions. Suitable non-proteinogenic amino acids may include 2-fluoro-phenylalanine, 2-methyl-tyrosine, and 2-naphthylalanine.

In some embodiments, the disclosed peptides comprise non-proteinogenic amino acids selected from nor-amino acids and/or linear core amino acids. Suitable nor-amino acids and/or linear core amino acids may include, but are not limited to norleucine (Nle), norvaline (Nva), 12-amino-dodecanoic acid, 8-amino-caprylic acid, 7-amino-enanthic acid, 6-amino-carpoic acid, and 5-amino-pentanoic acid.

In some embodiments, the disclosed peptides comprise non-proteinogenic amino acids which are coding, proteinogenic amino acids which are substituted with a substituent. Suitable non-proteinogenic amino acids may include may alanine which is substituted with a substituent selected from alkynyl (e.g., propargylglycine), azido (e.g., 4-azido-homo-alanine), thiophenyl, thienyl, (e.g., 3-(2-thineyl)-alanine), pyridyl (e.g., 3-(4-pyridyl-alanine (4-Pal)), anthrenyl, cycloalkyl, diphenyl, furyl, and naphthyl (e.g., 2-naphthylalanine).

In some embodiments, the disclosed peptides comprise non-proteinogenic amino acids comprising an ethylene-oxy moiety. Suitable non-proteinogenic amino acids may include amino acids having a formula NH₂—CH₂—CH₂—(O—CH₂—CH₂)ₙ—COOH, wherein n is selected from 1-24.

In some embodiments, the disclosed peptides comprise non-proteinogenic amino acids which are not α-amino acids. Suitable non-α-amino acids may include β-amino acids, α-amino acids, δ-amino acids, ε-amino acids, and ζ-amino acids (e.g., β-, γ-, δ, ε-, and ζ-amino acids of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine).

In some embodiments, the disclosed peptides comprise non-proteinogenic amino acids which are cycloamino acids (e.g., cycloamino acids other than proline). Cycloamino acids are amino acids which comprise a cyclic group formed by a nitrogen atom and a carboxyl group. Suitable cycloamino acids may include, but are not limited to aziridine-2-carboxylic acid, azetidine-2-carboxylic acid, piperidine-2-carboxylic acid, azepane-2-carboxylic acid, cycloleucine, homocycloleucine, 1-piperidine-4-carboxylic acid, piperidine-3-carboxylic acid, 1-piperazineacetic acid, 4-piperidineacetic acid, and 1-piperidineacetic acid.

In some embodiments, the disclosed peptides comprise proteinogenic amino acids and/or non-proteinogenic amino acids which optionally include an amino-protecting group. Suitable amino-protecting groups may include, but are not limited to allyloxycarbonyl (Alloc), 9-fluorenylmethyl carbonyl (Fmoc), t-butyl carbonyl (BOC), and benzyl carbonyl (Cbz).

The disclosed peptides may be conjugated directly to an antibody or an antigen-binding fragment thereof. In other embodiments, the disclosed peptides may be conjugated indirectly to an antibody or an antigen-binding fragment thereof via a linker. In some embodiments, the linker has a selected linear length. Suitable selected lengths may include at least about 5, 10, 15, 20, 25, 30, 35, 40, 45, 50 angstroms or longer, or a range bounded by any of these values (such as 5-10 angstroms, 10-20 angstroms, 15-20 angstroms, 15-25 angstroms, 20-35 angstroms, 30-40 angstroms, 35-40 angstroms, 35-50 angstroms, and 40-50 angstroms).

In some embodiments, the disclosed linkers have a spacer arm which may provide the linker with a selected length. For example, the disclosed linkers may have a formula represented as:

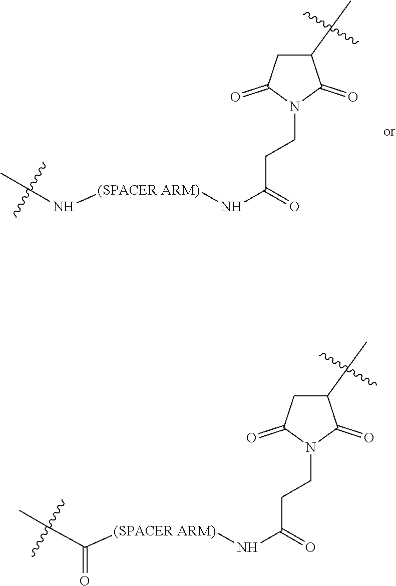

Suitable spacer arms may comprise a polymeric moiety such as polyethylene glycol.

In some embodiments, the linker has a formula selected from:

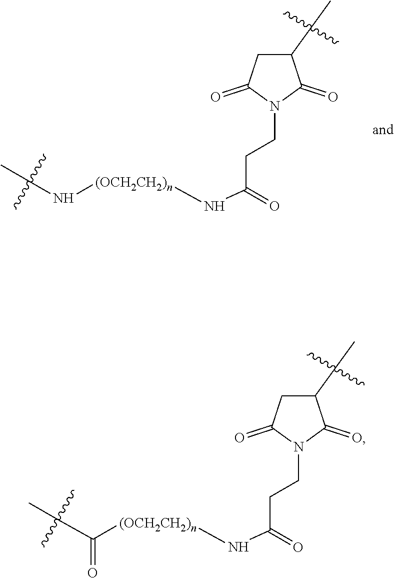

where n is an integer selected from 3-24.

As a particular embodiment, the linker may include a malemide moiety and a PEG moiety and may be referred to as a “maleimide-PEG linker” which conjugates an N-formyl-CF₃-methionine to an antibody. Exemplary conjugates may have formulas selected from:

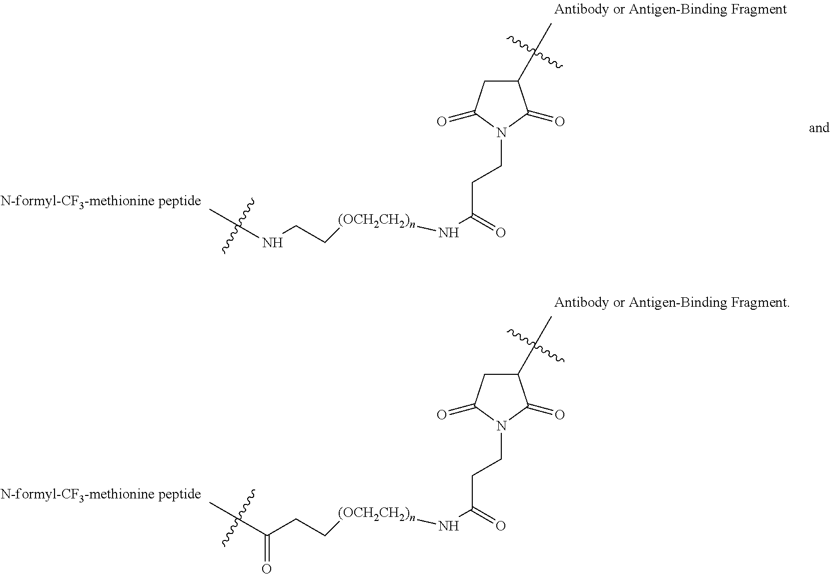

In some embodiments, the spacer arm comprises a peptide sequence of 1-20 amino acids. In some embodiments, the spacer arm is a peptide sequence comprising amino acids selected from glycine, serine, and alanine (e.g., (G₄S)ₘ where m is an integer selected from 1-5).

The disclosed linkers may comprise a polyethylene glycol moiety, for example, as a spacer arm or otherwise. In some embodiments, the linker comprises a polyethylene glycol (PEG) moiety (i.e., (—O—CH2-CH2)₁₋₂₄).

In some embodiments, the disclosed linkers may comprise two or more PEG moieties that are split by a non-PEG moiety such as amino acid moieties. In some embodiments, the disclosed linkers comprise a split polyethylene glycol moiety represented as -((PEG)₁₋₂₄)-(AA)₁₋₂-((PEG)₁₋₂₄)-, wherein AA is Glutamic acid bonded via gamma amino acylation or Lysine bonded via epsilon amino acylation.

Suitable peptide-linkers as disclosed herein may have a formula selected from:

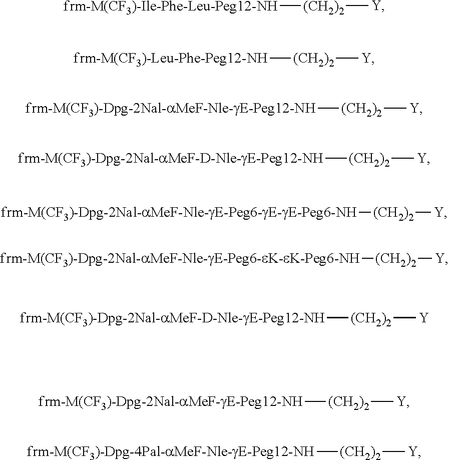

wherein Y comprises an amino group or a cysteine-reactive moiety for conjugating the peptide-linker to the antibody. Suitable cysteine-reactive moieties may include, but are not limited to maleimide, maleimide-diaminopropionic, iodoacetamide, or vinyl sulfone.

In some embodiments of the disclosed conjugates, the linker comprises a maleimide moiety and the linker is conjugated to the antibody or antigen-binding fragment thereof via a thioether bond formed between the maleimide moiety and a cysteine residue of the antibody.

Suitable amino acid residues for conjugating the disclosed peptides and linkers may include cysteine residues. Suitable cysteine residues may be engineered in the antibody or antigen-binding fragment thereof, where the cysteine residues are native cysteine residues of the antibody or antigen-binding fragment thereof or non-native cysteine residues of the antibody of antigen-binding fragment thereof. In some embodiments, the antibody or antigen-binding fragment thereof comprises an IgG heavy chain constant region and light chain region wherein said cysteine residue is selected from residue 124 of the C_{H}1 domain, residue 378 of the C_{H}3 domain, or both of residue 124 of the C_{H}1 domain and residue 378 of the C_{H}3 domain based on the EU Numbering Index. In some embodiments, the antibody or antigen-binding fragment thereof comprises an IgG heavy chain constant region comprising an isoleucine substituted at residue 247 for cysteine, a glutamic acid substituted at residue 332 for cysteine, or both of an isoleucine substituted at residue 247 for cysteine and a glutamic acid substituted at residue 332 for cysteine based on the EU Numbering Index. In some embodiments, the antibody comprises a heavy chain constant region comprising the amino acid sequence of SEQ ID NO: 12, 13, 14, 15, 16, 17, 18, 19, 20, 21, 52, 53, 54, 55, 56, or 57.

Suitable antibodies for the disclosed conjugates and methods may include human antibodies. Other suitable antibodies may include a mouse antibody, a rat antibody, or a rabbit antibody. Suitable therapeutic antibodies may include human antibodies, chimeric or hybrid antibodies, and humanized antibodies.

Suitable antibodies may include IgG isotypes. Suitable IgG isotypes may include isotypes having an IgG heavy chain constant region selected from human IgGI isotype or human IgG4 isotype.

Suitable antibodies may include monoclonal antibodies. Suitable antibodies may include monospecific antibodies and bispecific antibodies.

The disclosed peptides and optional linkers may be utilized to prepare conjugates of antibodies known in the art or antigen-binding fragments thereof. For example, the disclosed peptides and optional linkers may be conjugated to existing cancer therapeutic antibodies to prepare N-formyl-Met(CF₃) peptide-conjugated immunotherapeutics.

Exemplary cancer therapeutics for use in preparing peptide conjugates may include lgG1 therapeutic antibodies targeting solid tumors, including tumors expressing HER-2 (i.e, lgG1 antibodies such as trastuzumab and pertuzumab), liquid tumors, including liquid tumors expressing CD20 (i.e., lgG1 and lgG1-enhanced ADCC antibodies such as rituximab, ofatumumab, obinutuzumab, and AME133v) and antibodies targeting c-Met-expressing tumors (i.e., emibetuzumab).

The N-formyl-Met(CF₃)-peptides disclosed herein may be conjugated to therapeutic antibodies which comprise cytotoxic agents to function as additional therapeutic agents. Alternatively, the N-formyl-Met(CF₃)-peptides disclosed herein may replace cytotoxic agents in therapeutic antibodies to create novel therapeutic antibodies that target antigens overexpressed in cancer cells. Target antigens and representative therapeutic antibodies may include, but are not limited to, GPNMB (glembatumumab vedotin), CD56 (lorvotuzumab mertansine (IMGN-901)), TACSTD2 (TROP2; sacituzumab govitecan, (IMMU-132)), CEACAMS (labetuzumab SN-38), folate receptor-a (mirvetuximab soravtansine (IMGN-853), vintafolide), mucin 1, sialoglycotope CA6; SAR-566658, STEAP1 (vandortuzumab vedotin (RG-7450)), mesothelin (DMOT4039A, anetumab ravtansine (BAY-94-9343), BMS-986148), nectin 4 (enfortumab vedotin (ASG-22M6E); ASC-22CE), ENPP3 (AGS-16M8F), guanylyl cyclase C (indusatumab vedotin (MLN-0264)), SLC44A4 (ASG-5ME), NaPi2b, (lifastuzumab vedotin), CD70 (TNFSF7; DNIB0600A, AMG-172, MDX-1243, vorsetuzumab mafodotin (SGN-75)) CA9 carbonic anhydrase (BAY79-4620), 5T4 (TPBG; PF 06263507) SLTRK6 (ASG-15ME), SC-16 (anti-Fyn3; SC16LD6.5), tissue factor (HuMax-TF-ADC (TF-011-MMAE)), LIV-1 (ZIP6; SGN-LIVIA), P-Cadherin (PCA062) PSMA (MLN2704, PSMA-ADC), Fibronectin Extra-domain B (Human mAb L19 and F8), endothelin receptor ETB (RG-7636), VEGFR2 (CD309; anti-VEGFR-2ScFv-As203-stealth nanoparticles), Tenascin c (anti-InC-A1 antibody SIP(F16)), periostin (anti-periostin antibody), DLL3 (rovalpituzumab, soravtansine), HER 2 (T-DM1, ARX788, SYD985), EGFR (ABT-414, IMGN289 AMG-595), CD30 (brentuximab vedotin, iratumumab MDX-060), CD22 (Inotuzumab ozogamicin (CMC-544), pinatuzumab vedotin, epratuzumab SN38), CD79b (polatuzumab vedotin), CD19 (coltuximab ravtansine, SAR-3419, SGN-CD19A), CD138 (indatuximab ravtansine), CD74 (milatuzumab doxorubicin), CD37 (IMGN-529), CD33 (gemtuzumab ozogamicin, IMGN779, SGN CD33 A,) and CD98 (IGN523). (See, e.g., Thomas et al, Lancet Oncol. 2016 June; 17(6)e254-62 and Diamantis and Banerji, Brit. Journ. Cancer, 2016; 1 14, 362-367, the content of which is incorporated herein by reference in its entirety).

In some embodiments, the antibodies or antigen-binding fragments thereof of the disclosed conjugates comprise one or more of an HCDR1, an HCDR2, an HCDR3, an LCDR1, an LCDR2, and an LCDR3 of a known antibody, optionally selected from glembatumumab vedotin, lorvotuzumab mertansine (IMGN-901), TROP2; sacituzumab govitecan, (IMMU-132), labetuzumab SN-38, mirvetuximab soravtansine (IMGN-853), vintafolide, sialoglycotope CA6; SAR-566658, enfortumab vedotin (ASG-22M6E), ASC-22CE), ZIP6, SGN-LIVIA, DMOT4039A, anetumab ravtansine (BAY-94-9343), BMS-986148, sofituzumab vedotin, mirvetuximab soravtansine (IMGN-853), vintafolide, labetuzumab SN-38, glembatumumab vedotin, lorvotuzumab mertansine (IMGN-901), vandortuzumab vedotin (RG-7450), AGS-16M8F, indusatumab vedotin (MLN-0264), ASG-5ME, lifastuzumab vedotin, TNFSF7, DNIB0600A, AMG-172, MDX-1243, vorsetuzumab mafodotin (SGN-75), BAY79-4620, TPBG, PF 06263507, SLTRK6 (ASG-15ME), anti-Fyn3, SC16LD6.5), HuMax-TF-ADC (TF-011-MMAE), PCA062, Human mAb L19, Human mAb F8, RG-7636, CD309; anti-VEGFR-2ScFv-As203-stealth nanoparticles, anti-TnC-Al antibody SIP(F16), anti-periostin antibody, rovalpituzumab soravtansine, ABT-414, IMGN289 AMG-595, brentuximab vedotin, iratumumab MDX-060, inotuzumab ozogamicin (CMC-544), pinatuzumab vedotin, epratuzumab SN38, polatuzumab vedotin, coltuximab ravtansine, SAR-3419, SGN-CD19A, indatuximab ravtansine, milatuzumab doxorubicin, IMGN-529, gemtuzumab ozogamicin, IMGN779, SGN CD33 A, and IGN523.

The peptides and conjugates thereof may be formulated as pharmaceutical compositions. In some embodiments, the disclosed pharmaceutical compositions comprise: (i) a conjugated antibody or antigen-fragment thereof, as disclosed herein; and (ii) one or more pharmaceutically acceptable carriers, diluents, or excipients.

The disclosed peptides, conjugates, and pharmaceutical compositions thereof may be utilized in methods of treating diseases and disorders in a subject in need thereof. In some embodiments, the disclosed methods include methods of treating solid cancer or liquid tumors comprising administering to a patient in need thereof an effective amount of a conjugated antibody or a pharmaceutical composition thereof as disclosed herein. Suitable cancers for treating using the disclosed peptides, conjugates, pharmaceutical compositions, and methods may include, but are not limited to, breast cancer, lung cancer, prostate cancer, skin cancer, colorectal cancer, bladder cancer, kidney cancer, liver cancer, thyroid cancer, endometrial cancer, muscle cancer, bone cancer, mesothelial cancer, vascular cancer, fibrous cancer, leukemia, or lymphoma.

The disclosed peptides, conjugates, and pharmaceutical compositions may be utilized for therapy of a subject in need thereof. In some embodiments, the disclosed peptides, conjugates, and pharmaceutical compositions may be uses in the treatment of solid cancers or liquid tumors, which optionally are selected from breast cancer, lung cancer, prostate cancer, skin cancer, colorectal cancer, bladder cancer, kidney cancer, liver cancer, thyroid cancer, endometrial cancer, muscle cancer, bone cancer, mesothelial cancer, vascular cancer, fibrous cancer, leukemia or lymphoma.

Also disclosed herein are methods for activating neutrophils and in particular, for stimulating reactive oxygen species (ROS) production in neutrophils in vivo or in vitro. The methods comprise contacting neutrophils with conjugates as disclosed herein under conditions whereby the conjugates stimulate ROS production in neutrophils. In some embodiments, the conjugates comprise a spacer of a suitable length for inducing ROS production in neutrophils. For example, the conjugates may comprise a polyethylene glycol (PEG) spacer of a suitable length (e.g., a PEG spacer comprising at least 12 monomers) for inducing ROS production.

Also disclosed herein are compounds, which may be otherwise referred to herein

as peptides. In some embodiments, the compounds have a formula:
R—P¹—P²—P³—NH—(CH₂CH₂O)ₙ—CH₂CH₂—Y or a salt thereof, wherein:
R is a HC(═O)—
P¹ is Met(C(halogen)ₘ where m is 1-3 (e.g., Met(CF₃) or Met(CHF₂) or Met(CH₂F);
P² is 1-6 proteinogenic or non-proteinogenic amino acids bonded to P¹ and to each other via peptide bonds;
P³ is an amino acid comprising a side chain which comprises a —COOH moiety (e.g., glutamic acid or aspartic acid) or a —NH₂ moiety (e.g., lysine), optionally a glutamic acid residue connected through its side-chain gamma carboxyl group or a lysine residue connected through its side-chain epsilon amino group, and P³ is bonded to P² via a peptide bond;
n is an integer selected from 3-24; and
Y comprises amino or a cysteine reactive moiety, optionally wherein Y is selected from maleimide, maleimide-diaminopropionic, iodoacetamide, or vinyl sulfone.

In other embodiments, the disclosed compounds have a formula:
R—P¹—P²—P³—NH—(CH₂CH₂O)ₙ—CH₂CH₂—Y or a salt thereof, wherein:
R is a HC(═O)—
P¹ is Met or Met(C(halogen)ₘ where m is 1-3 (e.g., Met(CF₃) or Met(CHF₂) or Met(CH₂F);
P² is 1-6 proteinogenic or non-proteinogenic amino acids bonded to P¹ and to each other via peptide bonds;
P³ is an amino acid comprising a side chain which comprises a —COOH moiety (e.g., glutamic acid or aspartic acid) or a —NH₂ moiety (e.g., lysine), optionally a glutamic acid residue connected through its side-chain gamma carboxyl group or a lysine residue connected through its side-chain epsilon amino group, and P³ is bonded to P² via a peptide bond;
n is an integer selected from 3-24; and
Y comprises amino or a cysteine reactive moiety, optionally wherein Y is selected from maleimide, maleimide-diaminopropionic, iodoacetamide, or vinyl sulfone.

The disclosed peptides and conjugates preferably are agonists of one or more members of the family of formyl peptide receptors. Preferably, the disclosed peptides and conjugates are agonists of the formyl peptide receptor 1 (FPR-1). Preferably, the disclosed peptides and conjugates bind to one or more members of the family of formyl peptide receptors. Preferably, the disclosed peptides and conjugates bind to one or more members of the family of formyl peptide receptors present on the surface of neutrophils. Preferably, the disclosed peptides and conjugates bind to one or more members of the family of formyl peptide receptors with a Kd of at least about 10 uM, 1 uM, 100 nM, 50 nM, 10 nM or lower. As such, the disclosed peptides and conjugates may be utilized in methods for agonizing a formyl peptide receptor, the method comprising contacting the formyl peptide receptor with the peptides or conjugates.

### EXAMPLES

The following examples are illustrative and should not be interpreted to limit the scope of the claimed subject matter.

### Formyl-Met(CF₃) and PEG Peptides for Preparing Conjugates

### Abstract

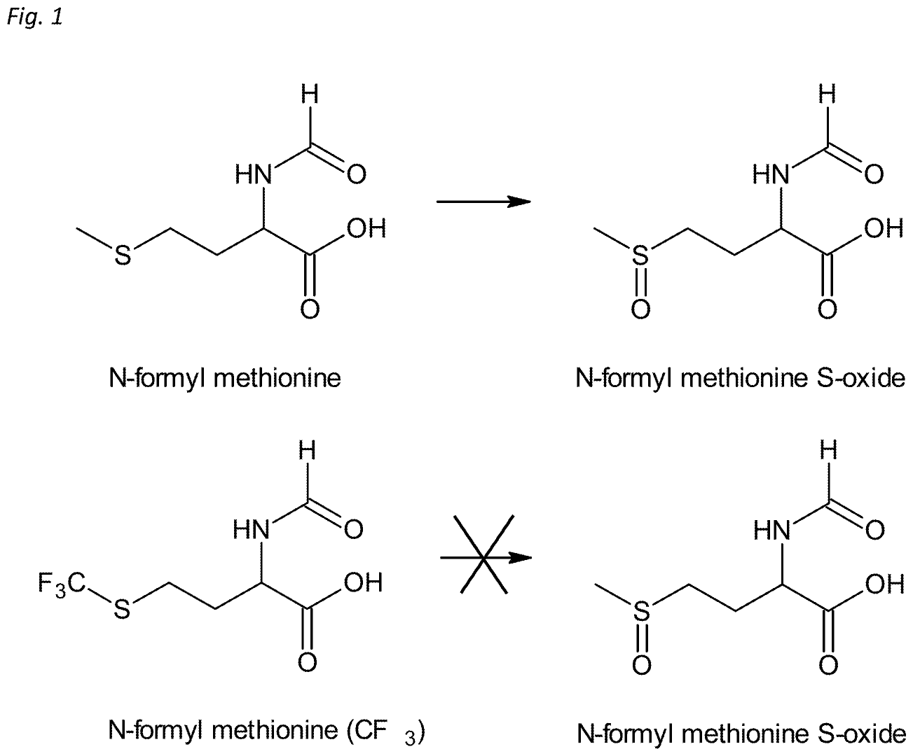

Native formyl-methionine peptides are subject to oxidation of the sulfur atom of the methionine residue and the formation of methyl-sulfoxide or generation of met(O) in vivo. (See FIG. 1). The oxidation of the sulfur atom of the methionine residue results in a significant reduction in formyl peptide receptor 1 (FPR-1) agonist activity (e.g., by as much as 10×). As such, formyl-trifluoro-methionine-based peptides (i.e., frm-Met(CF₃)-containing peptides) which are resistant to oxidation were prepared. Also prepared were peptides containing additional non-proteinogenic amino acids and polyethylene linker/spacers. The peptides were tested to determine whether they could function as FPR-1 agonists in order to determine if the peptides are suitable for preparing antibody bioconjugates to promote FPR-1-mediated and targeted cell killing by innate immune cells.

It was observed that the frm-Met(CF₃)-containing peptides are capable of activating the human formyl peptide receptor on neutrophils (FPR-1), which make them suitable for modifying antibodies and creating antibody conjugates that exhibit FPR-1-mediated and targeted cell killing. The further was observed that the linker length vis-à-vis polyethylene linker is important for inducing radical oxygen species (ROS) production in neutrophils. Finally, it was found that antibodies conjugated to frm-Met(CF₃)-containing peptides exhibiting a clearance profile similar to non-conjugated antibodies.

### Background

Bactabodies are antibody bioconjugates that engage the innate immune system in targeted cell killing. They consist of an antibody targeted to a specific cell that is conjugated to a pathogen associated molecular pattern (PAMP) that can activate innate immune cells to kill the target cell.

Formyl-peptides provide a PAMP that can be conjugated to an antibody to make a bactabody. Two well-characterized formyl peptides are formyl-Met-Leu-Phe (fMLF) and formyl-Met-Ile-Phe-Leu (fMIFL). fMLF and fMIFL are formyl peptide receptor (FPR) agonists, and FPR-1 receptors are activating receptors present on innate immune cells. fMLF is an effective agonist for human FPR-1, while fMIFL is an effective agonist for both human and mouse FPR-1 receptors.

One concern for in vivo activity of formyl-methionine based FPR-1 agonists is that oxidation of the methionine also reduces the activity of frm-Met peptides. (See FIG. 1). Also, although frm-Met peptides with native amino acids work well in vitro, they are rapidly degraded in vivo, presumably by native endopeptidases present on the surface of cells that bear FPR-1 or by circulating endopeptidases. To address metabolic stability, a modified FPR-1 agonist peptide containing non-proteinogenic acids that are stable for in vivo use was created. Finally, although frm-Met peptides function as agonists per se, the present work suggests that the frm-Met peptides must be displayed via a linker for maximum activity.

Described here are peptides having trifluoro modification of the methionine that eliminates the potential for methionine oxidation while retaining FPR-1 agonism. The disclosed peptides also include non-proteinogenic amino acids to inhibit digestion by endopeptidases. The disclosed peptides also include linkers comprising PEG, which the inventors show are important for maximum agonist activity

### Results and Observations

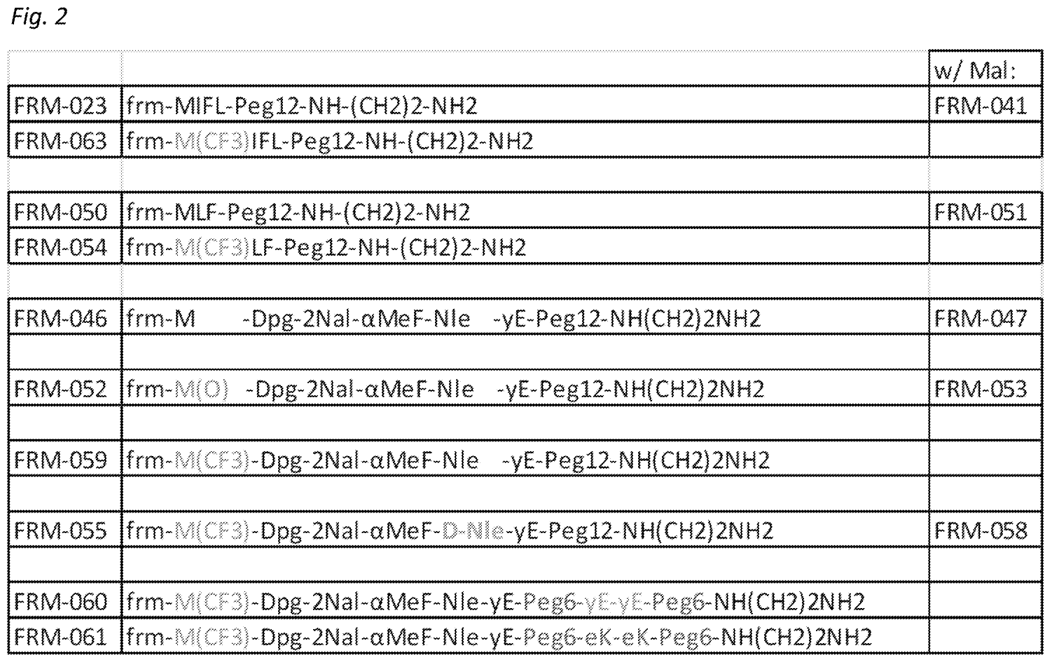

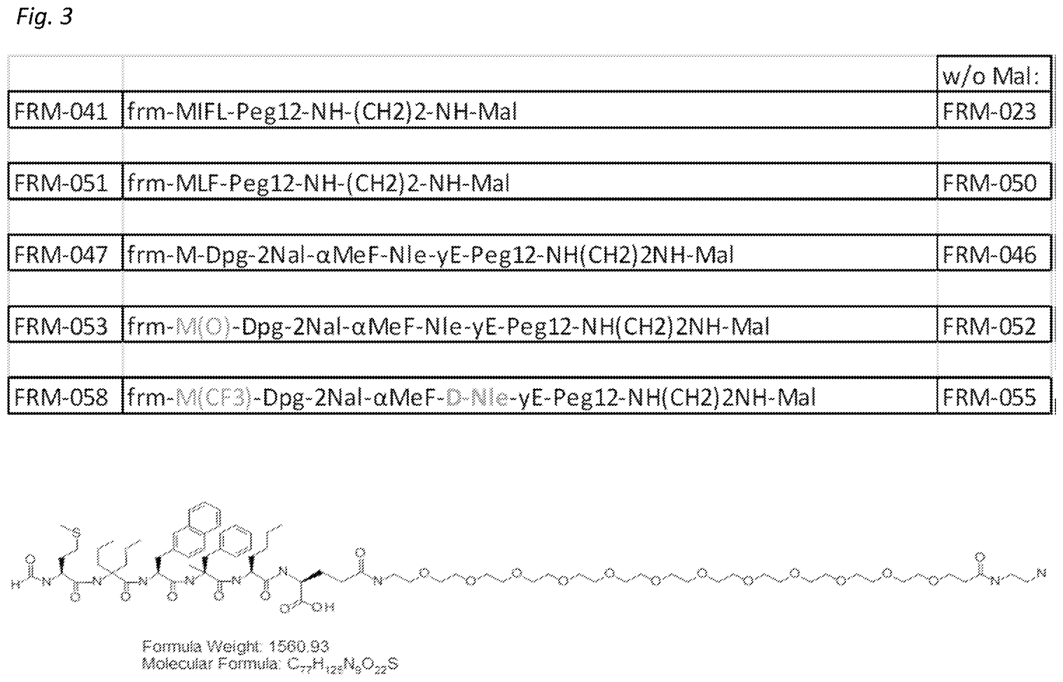

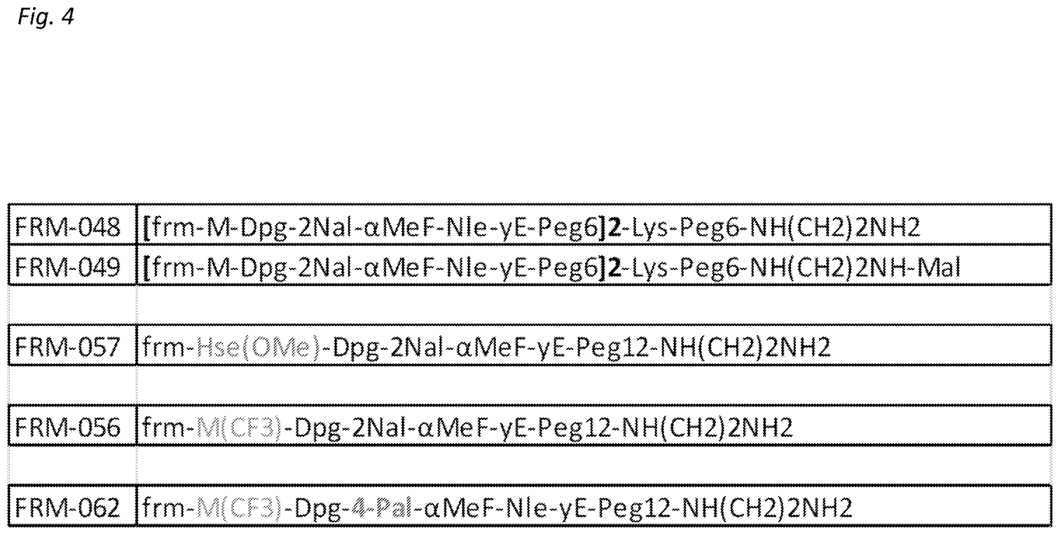

Creation of Peptides. As illustrated in FIG. 2, FIG. 3, and FIG. 4, the inventors prepared a panel of peptides having a frm-Met(CF₃), non-proteinogenic amino acids, and PEG linkers for in vitro and in vivo activity as agonists of FPR-1.

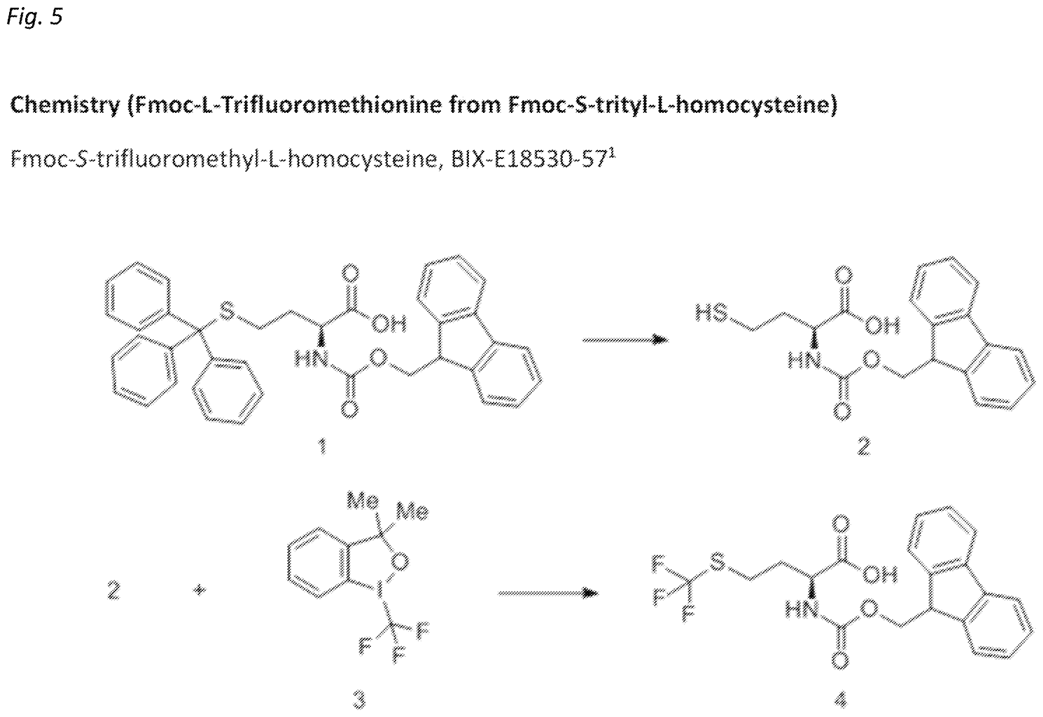

The synthesis chemistry for frm-Met(CF₃) is illustrated in FIG. 5. Fmoc-S-trityl-L-homocysteine (1, 1.695 g, 2.713 mmol) was dissolved DCM (25 mL) and triisopropylsilane (4 mL, 19.5 mmol) followed by TFA (15 mL, 198.4 mmol) were added at 21° C. and the reaction mixture was stirred for 1 h. Concentration in vacuo and co-distilled with MeOH to yield 2 (0.973 g, 2.72 mmol). 2 was dissolved in DCM (25 mL) and the solution cooled to −78° C. 3 (1.00 g, 2.94 mmol) dissolved in DCM was added the reaction mixture was stirred at −78° C.for 30 min. The reaction mixture was adsorbed on celite and purified via reverse phase chromatography (50% - 70% 0.1% FA in water/acetonitrile) to yield Fmoc-S-trifluoromethyl-L-homocysteine (4, 153 mg, 0.33 mmol, 12.2%). 1H NMR (500 HMz, DMSO) δ 12.7 (bs, 1H), 7.89 (d, 2H), 7.73-7.70 (m, 3H), 7.42 (t, 2H), 7.33 (t, 2H), 4.31-4.30 (m, 2H), 4.24-4.23 (m, 1H), 4.12-4.08 (m, 1H), 2.80-2.70 (m, 2H), 2.16-1.95 (m, 2H); ¹⁹F (352 MHZ, DMSO) δ −40 (s, 3F); MS [M⁺+Na] 448; purity 90% based on HPLC-UV (300 nm).

Briefly, FRM-023 and FRM063 comprise the human peptide MIFL and differ only in that FRM-063 comprises frm-Met(CF₃) while FRM-023 comprises frm-Met.

Similarly, FRM-050 and FRM-054 comprise the mouse peptide MFL and differ only in that FRM-054 comprises frm-Met(CF₃), while FRM-50 comprises frm-Met.

FRM-052 represents the oxidized control peptide.

FRM-059 represents a frm-Met(CF₃) derivative of FRM-047.

FRM-055 represents a D-Nle derivative of FRM-047.

FRM-060 and FRM-061 include two “split” PEG6 linkers in order to reduce flexibility of the PEG12 linker present in FRM-059.

FRM-041 and FRM-051 are versions of FRM-023 and FRM-050, respectively, having a terminal maleimide.

FRM-053 is a version of FRM-052 having a terminal maleimide.

FRM-058 is a version of FRM-055 having a terminal maleimide.

FRM-048 and FRM-049 are branched versions of FRM-047 comprising two peptides and either lacking a terminal maleimide (FRM-048) or having a terminal maleimide (FRM-049).

FRM-057 is a derivative of FRM-047 having an N-terminal methoxinine residue (i.e., methionine in which the sulfur atom is replaced with an oxygen atom).

FRM-056 is a derivative of FRM-047 having a frm-Met(CF₃) and lacking a Nle.

FRM-062 is a derivative of FRM-047 having a frm-Met(CF₃) and a 4-Pal.

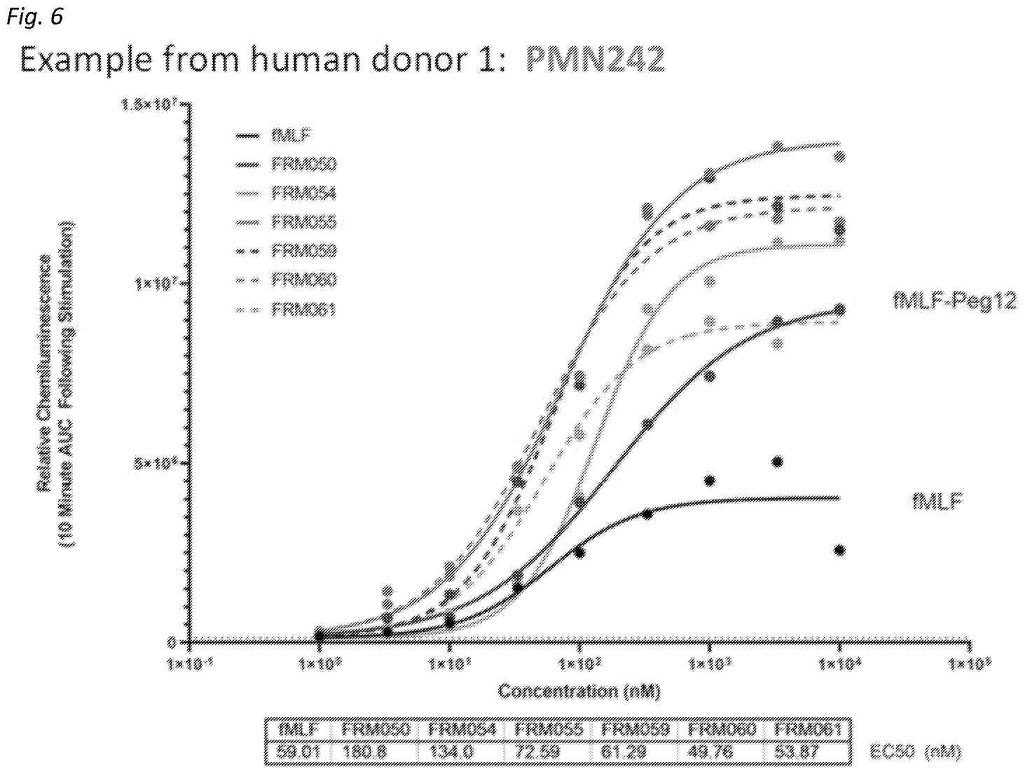

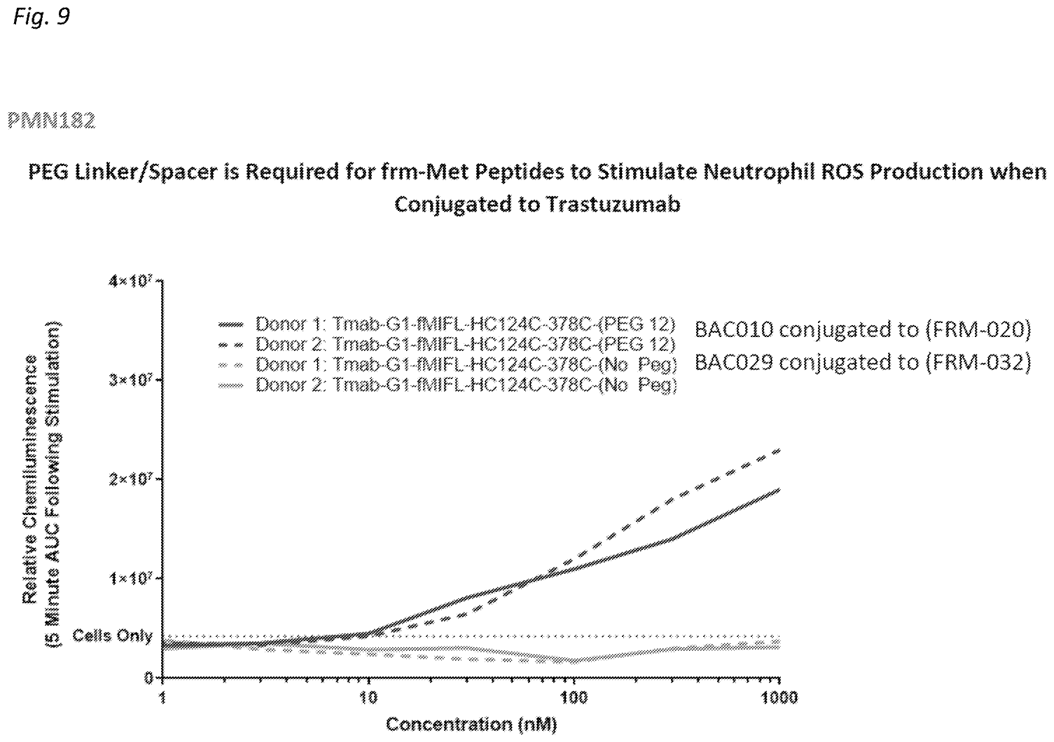

In vitro. C-Terminal Pegylation of frm-Met peptides and frm-Met(CF₃) peptides enhances FPR-1 mediated reactive oxygen species (ROS) production from primary human neutrophils. As illustrated in FIG. 6 and FIG. 9, C-terminal pegylation of frm-Met peptides and frm-Met(CF₃) peptides enhanced activity for ROS production in neutrophils. ROS production from primary human neutrophils also was enhanced with frm-Met(CF₃) peptides having non-proteinogenic amino acids (plus PEG) compared to frm-Met(CF₃) peptides with proteinogenic amino acids (plus PEG).

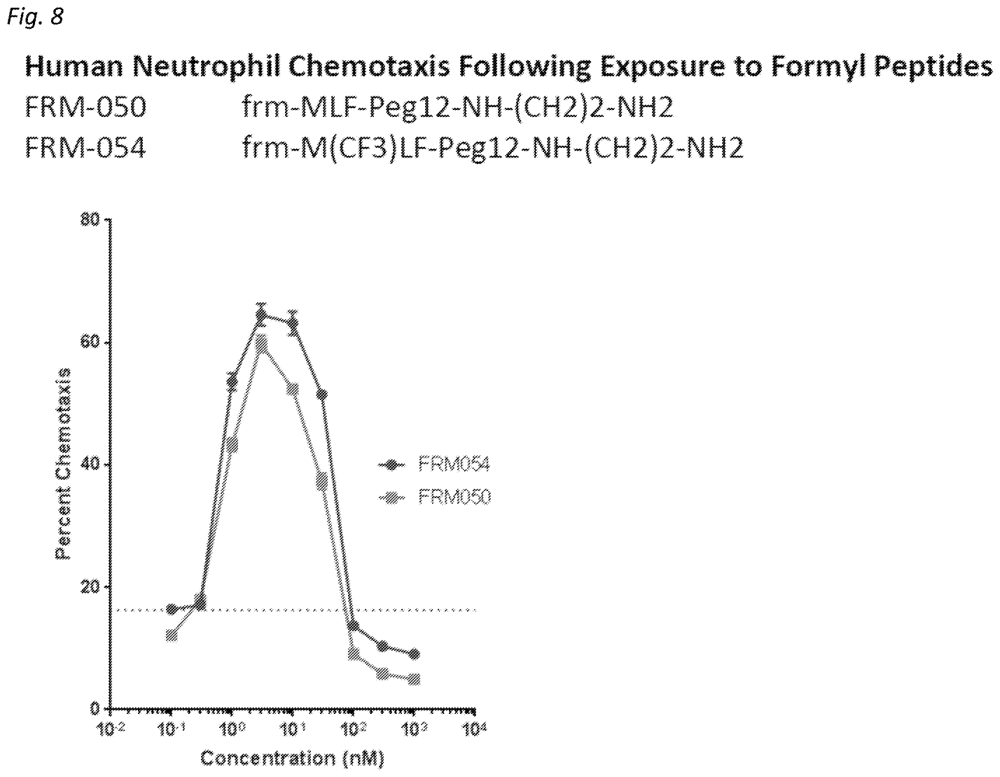

As illustrated in FIG. 8, migration of primary human neutrophils was similar between frm-Met peptides with proteinogenic amino acids (MLF plus PEG) and frm-Met(CF₃) peptides with proteinogenic amino acids (frm-M(CF₃)LF plus PEG).

The inventors also tested conjugated peptides. As illustrated in FIG. 9, ROS production from primary human neutrophils by peptides conjugated to specific eCys sites on trastuzumab by maleimide requires a linker/spacer (e.g., a PEG linker/spacer). frm-Met peptides conjugated to trastuzumab and lacking a spacer were incapable of stimulating ROS production from human neutrophils.

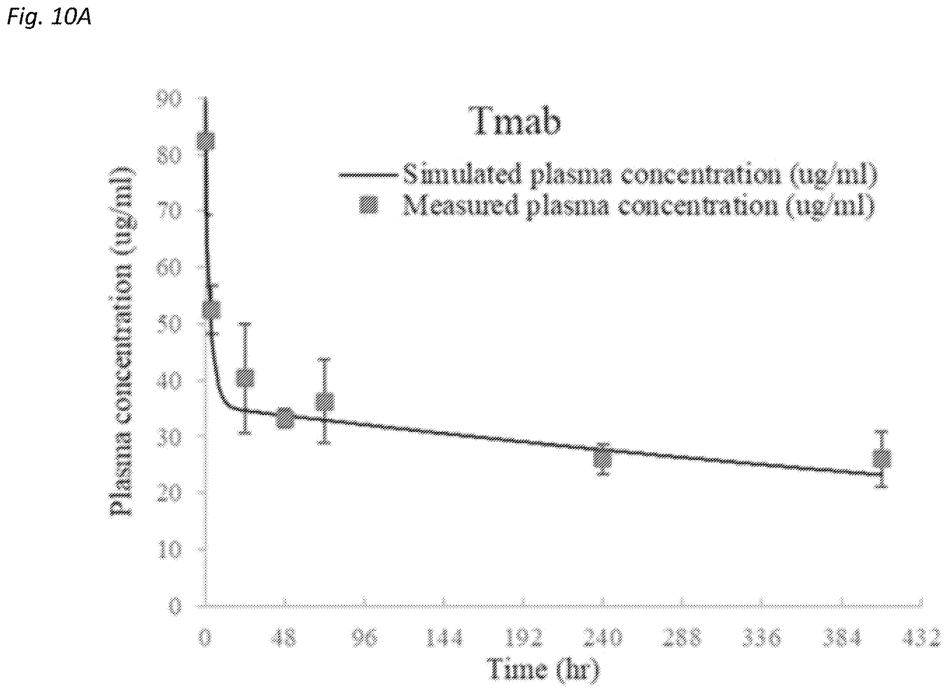

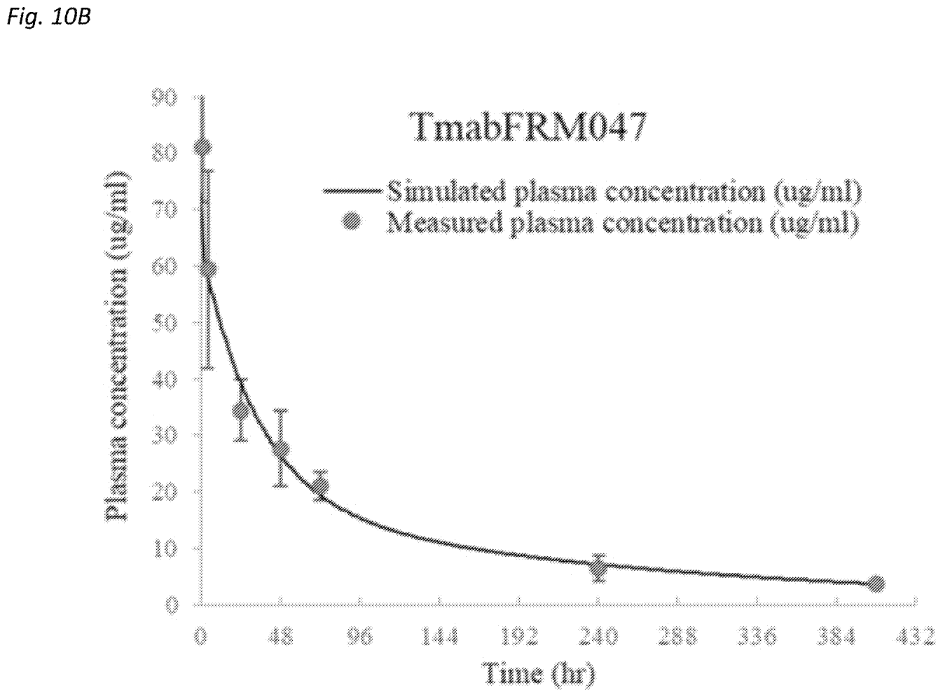

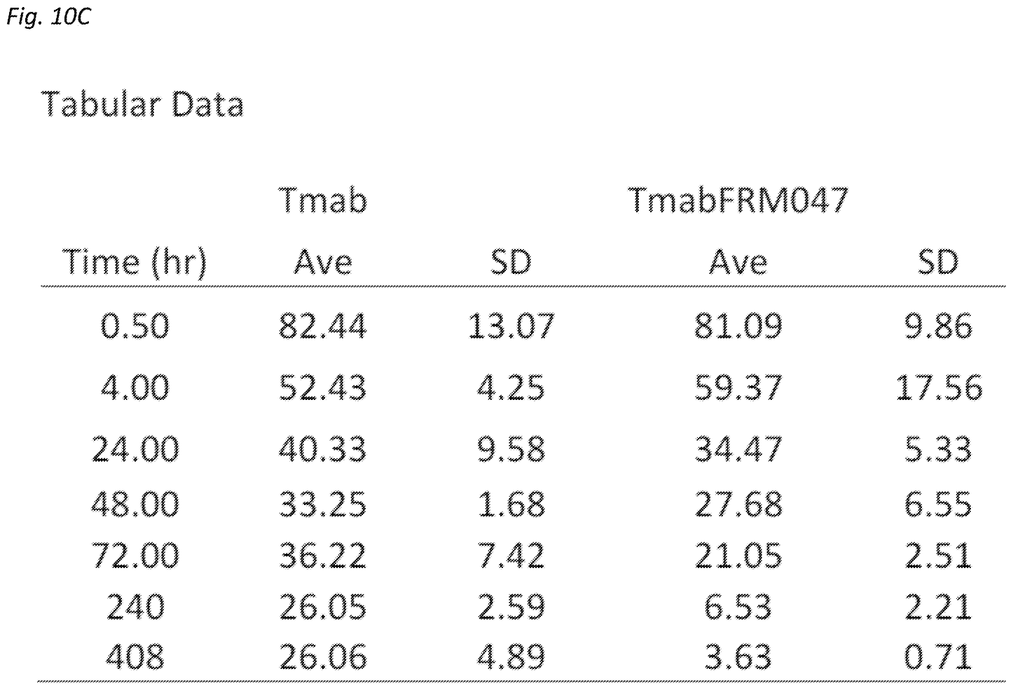

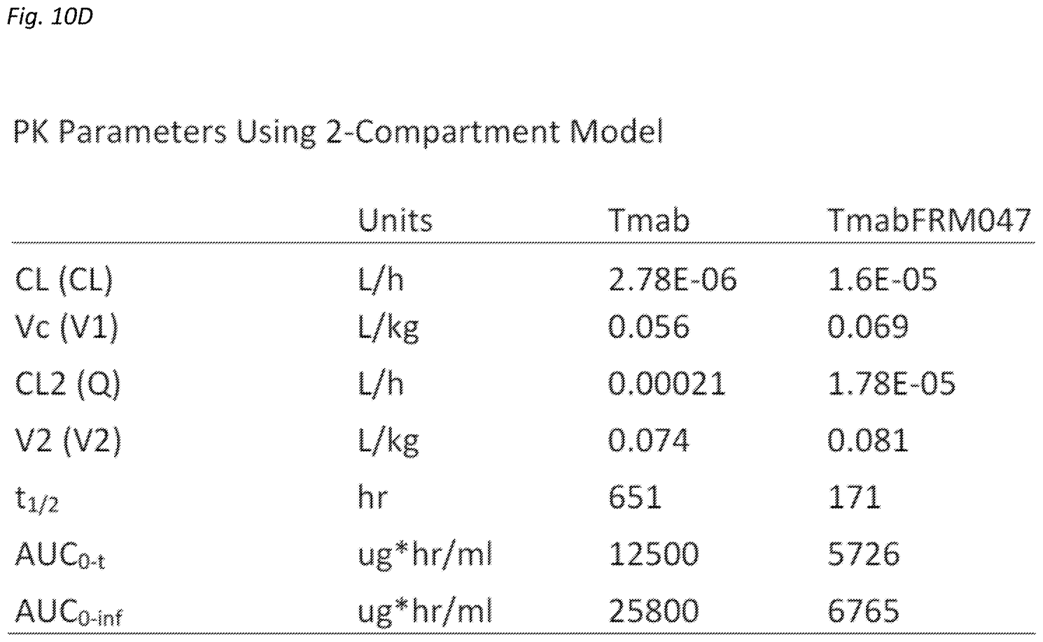

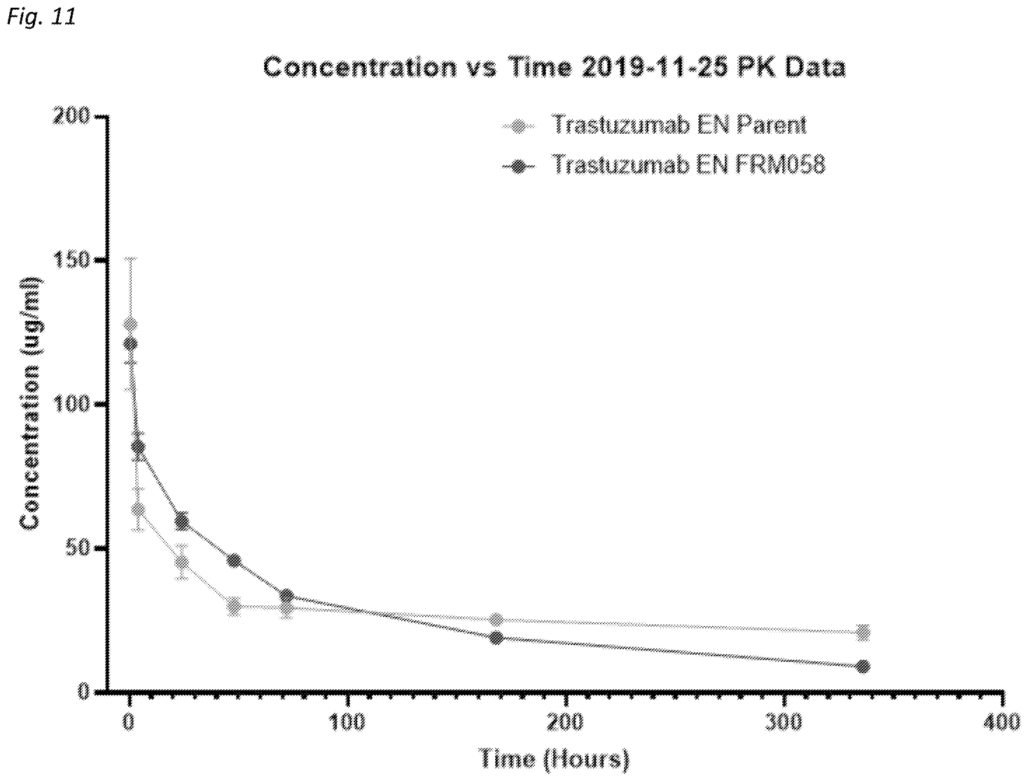

In vivo. Trastuzumab bactabodies with frm-Met peptides and non-proteinogenic amino acids clear faster than trastuzumab parent antibody, resulting in lower exposure with bactabody. As illustrated in FIG. 10A versus FIG. 10B (and FIG. 10C and FIG. 10D), conjugating peptide FRM-047 resulted in higher clearance for conjugated antibody versus the non-conjugated parent antibody. Estimated exposure based on AUCO-oo show 3.8-fold greater exposure for Tmab parent compared to Tmab bactabody with FRM-047 peptide. However, clearance was similar to parent trastuzumab when trastuzumab (Tmab) was conjugated to peptide FRM-058, which is a derivative of peptide FRM-047 having a frm-Met(CF₃) moiety. (See FIG. 11). Thus, trifluoro substitution on frm-Met(CF₃) the trastuzumab bactabody provides reduced clearance versus the equivalent bactabody with the frm-Met peptide and clearance is similar to the Tmab parent antibody.

### Methods

ROS Production. Human neutrophils were purified from fresh blood draws as previously described. Production of reactive oxygen species by primary human neutrophils was measured using luminol-amplified chemiluminescence. Following isolation, PMNs were suspended at 1×10⁶ cells/ml in HBSS containing calcium and magnesium (Gibco #14025-092) supplemented with 0.25% human serum albumin (Gemini Bio products #800-120) and 50 μM Luminol (Sigma-Aldrich #123072-2.5G). 100 μl of cell suspension (1×10⁵ total cells) was then distributed into each well of a 96-well plate suitable for fluorescence measurement (Greiner #655098) and temperature equilibrated to 37° C. for 5 minutes. Following equilibration, 10× solutions of agonists were applied to the wells, achieving a 1× final concentration. Immediately after the addition of agonists, chemiluminescence signal was recorded by luminometer. Area under the curve (AUC) scores were calculated using luminescence signal from the first 5 minutes of each run. Values are displayed relative luminescence units.

Chemotaxis. Neutrophil chemotaxis across transwell membranes (Corning #3415) towards agonists in a modified Boyden chamber assay was measured. Approximately 2-4×10⁵ cells from neutrophil-enriched preparations were seeded in upper transwell chambers on membranes with 3.0 □m pores. The lower transwell chambers contained buffer with or without test agents. Following seeding in transwells, cells were placed at 37° C. in a humidified incubator. After one hour any cells in the upper chamber were removed, and the percentage of cells which successfully migrated to the lower chamber was quantified using CellTiter-Glo™ (Promega #G7571) according to manufacturer specified protocol. Percent of successful migration relative to maximal cell-input values were determined using standard curves. Values are displayed as percent of starting cell-input. The U-shaped dose response curve is anticipated. Migration from upper to lower chamber requires a concentration gradient, and as concentration of peptide increases, enhanced diffusion of peptide into the upper chamber leads to deterioration of the concentration gradient necessary to drive migration from upper to lower chamber.

### Conclusion

Pegylation of frm-Met peptides and frm-Met(CF₃) peptides enhances FPR-1 mediated reactive oxygen species (ROS) production from primary human neutrophils, frm-Met(CF₃) peptides with non-proteinogenic amino acids are more effective than frm-Met(CF₃) peptides with native amino acids at driving FPR-1 mediated ROS production from primary human neutrophils

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

Certain embodiments are below, numbered 1-53:

(1) A conjugated antibody or antigen-binding fragment thereof comprising an antibody or an antigen-binding fragment thereof that is conjugated to a peptide comprising an N-formyl-halogenated methionine residue at the N-terminus of the peptide.

(2) The conjugated antibody of embodiment 1, wherein the N-formyl-halogenated methionine residue is N-formyl-trifluorinated methionine.

(3) The conjugated antibody of embodiment 1 or 2, wherein the antibody is conjugated to the peptide via a linker.

(4) The conjugated antibody of any of the foregoing embodiments, wherein the conjugated antibody has a formula represented as:

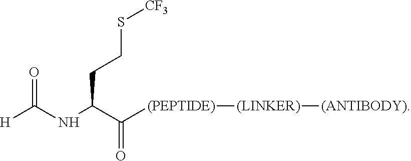

(5) The conjugated antibody of any of the foregoing embodiments, wherein the peptide comprises a C-terminal glutamic acid residue and the peptide is conjugated to the linker via an amide bond formed between the gamma carboxyl group of the glutamic acid and an amino group of the linker.

(6) The conjugated antibody of any of embodiments 1-5, wherein the peptide comprises a C-terminal lysine residue and the peptide is conjugated to the linker via an amide bond formed between the epsilon amino group of the lysine and a carboxyl group of the linker.

(7) The conjugated antibody of any of the foregoing embodiments, wherein the conjugated antibody has a formula represented as:

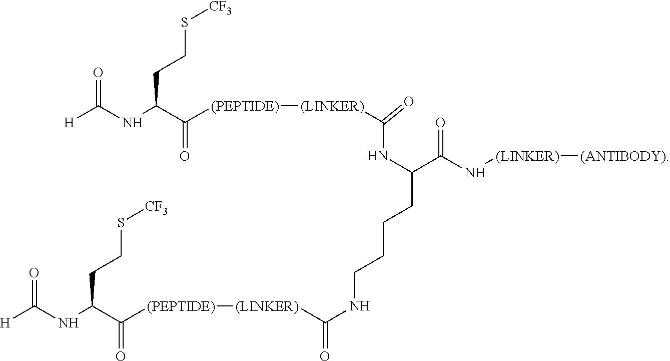

(8) The conjugated antibody of any of the foregoing embodiments, wherein the peptide comprises 2-10 amino acids, which optionally are non-proteinogenic amino acids and include a halogen-substituted amino acid (e.g., N-formyl-trifluorinated methionine at the N-terminus).

(9) The conjugated antibody of any of the foregoing embodiments, wherein the peptide comprises 2-10 amino acids, which optionally are non-proteinogenic amino acids selected from D-amino acids (e.g., D-amino acids of alanine, arginine, asparagine, aspartic acid, cysteine, glutamic acid, glutamine, histidine, isoleucine, leucine, lysine, methionine,phenylalanine, proline, serine, threonine, tryptophan, tyrosine, and valine).

(10) The conjugated antibody of any of the foregoing embodiments, wherein the peptide comprises 2-10 amino acids, which optionally are non-proteinogenic amino acids selected from homologues of amino acids lacking one or more methylene groups between the α-carbon and the side chain or homologous of amino acids possessing an additional methylene group between the α-carbon and the side chain (e.g., homo-amino acids, bishomo-amino acids, and the like).

(11) The conjugated antibody of any of the foregoing embodiments, wherein the peptide comprises 2-10 amino acids, which optionally are non-proteinogenic amino acids selected from homologues of amino acids lacking one or more methylene groups between the α-carbon and the side chain (e.g., 2-aminoisobutyric acid, 2-amino-2-hydroxyacetic acid, 2α-methyl-2-hydroxy-glycine, 2-amino-2-methylbutyric acid (i.e., isovaline), methylcysteine, azetidine-2-carboxylic acid, phenylglycine, 4-hydroxyphenylglycine, 3-indolylglycine, aminomalonic acid, 2,3-diamino-3-oxopropanoic acid, 2-amino-2-(1H-imidazol-5-yl)acetic acid, ornithine, 2,4-diaminobutanoic acid, 2,3-diaminopropionic acid, and 2-amino-4-(diaminomethylideneamino)butanoic acid).

(12) The conjugated antibody of any of the foregoing embodiments, wherein the peptide comprises 2-10 amino acids, which optionally are non-proteinogenic amino acids selected from homo-amino acids (e.g., homo-amino acids of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine, such as homo-alanine, homo-arginine, homo-glutamine, homo-glutamic acid, homo-isoleucine, homo-leucine, homo-lysine, homo-methionine, homo-phenylalanine, homo-proline (i.e., piperidine-2-carboxylic acid), homo-serine, homo-threonine, homo-tryptophan, and homo-tyrosine).

(13) The conjugated antibody of any of the foregoing embodiments, wherein the peptide comprises 2-10 amino acids, which optionally are non-proteinogenic amino acids selected from bishomo-amino acids (e.g., bishomo-amino acids of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine).

(14) The conjugated antibody of any of the foregoing embodiments, wherein the peptide comprises 2-10 amino acids, which optionally are non-proteinogenic amino acids selected from alkylated amino acids comprising an alkyl substitution (e.g., a C₁-C₆ alkyl substitution) on the α-carbon (e.g., α-carbon, alkyl substituted alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine, such as 2-methyl-serine (i.e., α-methyl-serine), 2-methyl-threonine (i.e., α-methyl-threonine), α-methyl-valine, α-methyl-leucine, 2-amino-2,3-dimethyl-pentanoic acid (i.e., α-methyl-isoleucine), α-methyl-methionine, α-methyl-cysteine, 2-methyl-proline, α-methyl-phenylalanine, α-methyl-tyrosine, α-methyl-tryptophan, 2-methyl-aspartic acid, 2-methyl-glutamic acid, 2,4-diamino-2-methyl-4-oxobutanoic acid (i.e., α-methyl-asparagine), 2,5-diamino-2-methyl-5-oxopentanoic acid (i.e., α-methyl-glutamine), α-methyl-histidine, α-methyl-lysine, and 2-methyl-arginine (i.e., α-methyl-lysine)).

(15) The conjugated antibody of any of the foregoing embodiments, wherein the peptide comprises 2-10 amino acids, which optionally are non-proteinogenic amino acids selected from di-alkylated amino acids comprising a di-alkyl substitution (e.g., a C₁-C₆ alkyl substitution) on the α-carbon (e.g., α-carbon, di-alkyl substituted glycine, such as dipropylglycine (Dpg)).

(16) The conjugated antibody of any of the foregoing embodiments, wherein the peptide comprises 2-10 amino acids, which optionally are non-proteinogenic amino acids selected from alkylated amino acids comprising an alkyl substitution (e.g., a C₁-C₆ alkyl substitution) on the amino group (e.g., N-alkylated alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine, such as N-methyl-alanine, N-methyl-arginine, N-methyl-asparagine, N-methyl-aspartic acid, N-methyl-cysteine, N-methyl-glutamic acid, N-methyl-glutamine, N-methyl-glycine, N-methyl-histidine, N-methyl-isoleucine, N-methyl-leucine, N-methyl-lysine, N-methyl-methionine, N-methyl-phenylalanine, N-methyl-proline, N-methyl-serine, N-methyl-threonine, N-methyl-tryptophan, N-methyl-tyrosine, and N-methyl-valine.

(17) The conjugated antibody of any of the foregoing embodiments, wherein the peptide comprises 2-10 amino acids, which optionally are non-proteinogenic amino acids selected from phenylalanine, tyrosine, tryptophan, histidine, proline, naphthylalanine, optionally comprising one or more ring-substitutions selected from C₁-C₆ alkyl substitutions, halogen-substitutions, and cyano-substitutions (e.g., 2-fluoro-phenylalanine, 2-methyl-tyrosine, and 2-naphthylalanine).

(18) The conjugated antibody of any of the foregoing embodiments, wherein the peptide comprises 2-10 amino acids, which optionally are non-proteinogenic amino acids selected from nor-amino acids and/or linear core amino acids such as norleucine (Nle), norvaline (Nva), 12-amino-dodecanoic acid, 8-amino-caprylic acid, 7-amino-enanthic acid, 6-amino-carpoic acid, and 5-amino-pentanoic acid.

(19) The conjugated antibody of any of the foregoing embodiments, wherein the peptide comprises 2-10 amino acids, which optionally are non-proteinogenic amino acids selected from amino acids (e.g., alanine) comprising a substitution selected from alkynyl (e.g., propargylglycine), azido (e.g., 4-azido-homo-alanine), thiophenyl, thienyl, (e.g., 3-(2-thineyl)-alanine), pyridyl (e.g., 3-(4-pyridyl-alanine (4-Pal)), anthrenyl, cycloalkyl, diphenyl, furyl, and naphthyl.

(20) The conjugated antibody of any of the foregoing embodiments, wherein the peptide comprises 2-10 amino acids, which optionally are non-proteinogenic amino acids selected from amino acids having a formula NH₂—CH₂—CH₂—(O—CH₂—CH₂)ₙ—COOH, wherein n is selected from 1-24.

(21) The conjugated antibody of any of the foregoing embodiments, wherein the peptide comprises 2-10 amino acids, which optionally are non-proteinogenic amino acids selected from β-amino acids, γ-amino acids, δ-amino acids, ε-amino acids, and ζ-amino acids (e.g., β-, γ-, δ, ε-, and ζ-amino acids of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine).

(22) The conjugated antibody of any of the foregoing embodiments, wherein the peptide comprises 2-10 amino acids, which optionally are non-proteinogenic amino acids selected from cycloamino acids (e.g., aziridine-2-carboxylic acid, azetidine-2-carboxylic acid, piperidine-2-carboxylic acid, azepane-2-carboxylic acid, cycloleucine, homocycloleucine, 1-piperidine-4-carboxylic acid, piperidine-3-carboxylic acid, 1-piperazineacetic acid, 4-piperidineacetic acid, and 1-piperidineacetic acid).

(23) The conjugated antibody of any of the foregoing embodiments, wherein the peptide comprises an amino-protecting group (—N— optionally selected from allyloxycarbonyl (Alloc), 9-fluorenylmethyl carbonyl (Fmoc), t-butyl carbonyl (BOC), and benzyl carbonyl (Cbz).

(24) The conjugated antibody of any of the foregoing embodiments, wherein the linker comprises a spacer arm having a length of about 10-50 angstroms (or 10-40 angstroms, or 10-30 angstroms, or 10-20 angstroms).

(25) The conjugated antibody of any of the foregoing embodiments, wherein the linker comprises a spacer arm and the linker has a formula selected from:

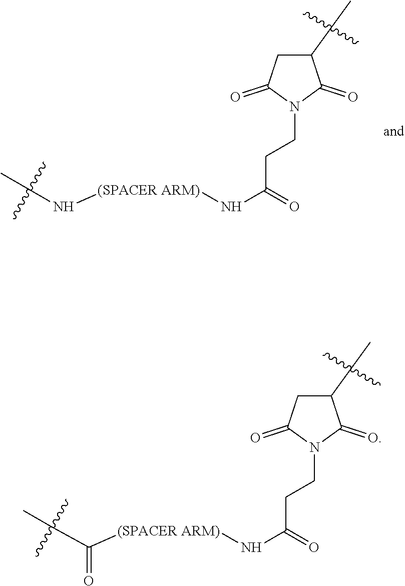

(26) The conjugated antibody of embodiment 24 or 25, wherein the linker has a formula selected from

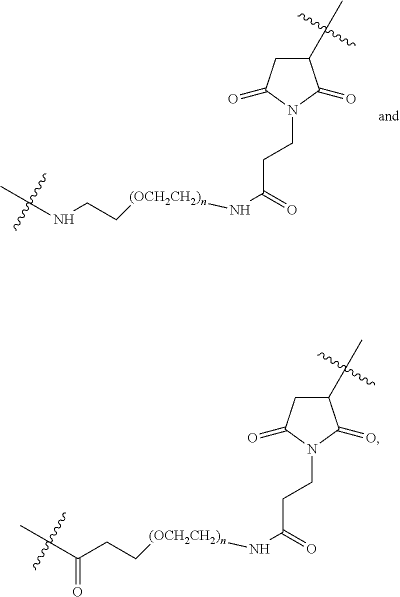

where n is an integer selected from 3-24.

(27) The conjugated antibody of embodiment 24 or 25, wherein the spacer arm is a peptide sequence comprising amino acids selected from glycine, serine, and alanine (e.g., (G₄S)ₘ where m is an integer selected from 1-5).

(28) The conjugated antibody of any of the foregoing embodiments, wherein the linker comprises a polyethylene glycol (Peg) moiety (i.e., (—O—CH2-CH2)₁₋₂₄).

(29) The conjugated antibody of any of the foregoing embodiments, wherein the linker comprises a split polyethylene glycol moiety represented as -((Peg)₁₋₂₄)-(AA)₁₋₂-((Peg)₁₋₂₄)-, wherein AA is Glutamic acid residue connected through its side-chain gamma carboxyl group or Lysine residue connected through its side-chain epsilon amino group.

(30) The conjugated antibody of any of the foregoing embodiments, wherein the peptide-linker has a formula selected from: frm-M(CF₃)-Ile-Phe-Leu-Peg12-NH—(CH₂)₂—Y, frm-M(CF₃)-Leu-Phe-Peg12-NH—(CH₂)₂—Y, frm-M(CF₃)-Dpg-2Nal-αMeF-Nle-γE-Peg12-NH—(CH₂)₂—Y, frm-M(CF₃)-Dpg-2Nal-αMeF-D-Nle-E-Peg12-NH—(CH₂)₂—Y, frm-M(CF₃)-Dpg-2Nal-αMeF-Nle-γE-Peg6-γE-γE-Peg6-NH—(CH₂)₂—Y, frm-M(CF₃)-Dpg-2Nal-αMeF-Nle-γE-Peg6-εK-εK-Peg6-NH—(CH₂)₂—Y, frm-M(CF₃)-Dpg-2Nal-αMeF-D-Nle-γE-Peg12-NH—(CH₂)₂—Y, frm-M(CF₃)-Dpg-2Nal-αMeF-γE-Peg12-NH—(CH₂)₂—Y, and frm-M(CF₃)-Dpg-4Pal-αMeF-Nle-γE-Peg12-NH—(CH₂)₂—Y, wherein Y comprises an amino group or a cysteine-reactive moiety for conjugating the peptide-linker to the antibody.

(31) The conjugated antibody of embodiment 30, wherein the cysteine-reactive moiety is selected from maleimide, maleimide-diaminopropionic, iodoacetamide, or vinyl sulfone.

(32) The conjugated antibody of any of the foregoing embodiments, wherein the linker comprises a maleimide moiety and the linker is conjugated to the antibody via a thioether bond formed between the maleimide moiety and a cysteine residue of the antibody.

(33) The conjugated antibody of embodiment 32, wherein the antibody comprises an IgG heavy chain constant region and light chain region wherein said cysteine residue is selected from residue 124 of the C_{H}1 domain, residue 378 of the C_{H}3 domain, or both of residue 124 of the C_{H}1 domain and residue 378 of the C_{H}3 domain.

(34) The conjugated antibody of any of the foregoing embodiments, wherein the antibody is a human antibody, a chimeric or hybrid antibody, or a humanized antibody.

(35) The conjugated antibody of any of the foregoing embodiments, wherein the antibody comprises an IgG heavy chain constant region selected from human IgGI isotype or human lgG4 isotype.

(36) The conjugated antibody of any of the foregoing embodiments, wherein the antibody comprises an IgG heavy chain constant region comprising an isoleucine substituted at residue 247, a glutamic acid substituted at residue 332, or both of an isoleucine substituted at residue 247 and a glutamic acid substituted at residue 332, with numbering based on EU Index Numbering.

(37) The conjugated antibody of any of the foregoing embodiments, wherein the antibody comprises a heavy chain constant region comprising the amino acid sequence of SEQ ID NO: 12, 13, 14, 15, 16, 17, 18, 19, 20, 21, 52, 53, 54, 55, 56, or 57.

(38) The conjugated antibody of any of the foregoing embodiments, wherein the antibody is a monoclonal antibody.

(39) The conjugated antibody of any of the foregoing embodiments, wherein the antibody is a bispecific antibody.

(40) The conjugated antibody of any of the foregoing embodiments, wherein the antibody binds to an antigen selected from HER2, PSMA, TROP2, MUC-1, Nectin 4, LIV-1, mesothelin, MUC-16, folate receptor-R1, CEACAM5, GPNMB, CD56, STEAP1, ENPP3, guanylyl cyclase C, SLC44A4, NaPi2b, CD70, CA9 carbonic anhydrase, 5T4, SC-16, tissue factor, P-Cadherin, Fibronectin Extra-domain B, endothelin receptor ETB, VEGFR2, Tenascin c, periostin, DLL3, EGFR, CD30, CD22, CD79b, CD19, CD138, CD74, CD37, CD33, and CD98.

(41) The conjugated antibody of any of the foregoing embodiments, wherein the antibody comprises one or more of an HCDRI, an HCDR2, an HCDR3, an LCDR1, an LCDR2, and an LCDR3 of an antibody selected from T-DM1, ARX788, SYD985, MLN2704, PSMA-ADC, TACSTD2, sacituzumab govitecan, (IMMU-132)), mucin 1, sialoglycotope CA6; SAR-566658, enfortumab vedotin (ASG-22M6E), ASC-22CE), ZIP6, SGN-LIVIA, DMOT4039A, anetumab ravtansine (BAY-94-9343), BMS-986148), sofituzumab vedotin, mirvetuximab soravtansine (IMGN-853), vintafolide, labetuzumab SN-38, glembatumumab vedotin, lorvotuzumab mertansine (IMGN-901), vandortuzumab vedotin (RG-7450), AGS-16M8F, indusatumab vedotin (MLN-0264), ASG-5ME, lifastuzumab vedotin, TNFSF7, DNIB0600A, AMG-172, MDX-1243, vorsetuzumab mafodotin (SGN-75), BAY79-4620, TPBG, PF 06263507, SLTRK6 (ASG-15ME), anti-Fyn3, SC16LD6.5), HuMax-TF-ADC (TF-011-MMAE), PCA062, Human mAb L19 and F8, RG-7636, CD309; anti-VEGFR-2ScFv-As2O3-stealth nanoparticles, anti-TnC-A1 antibody SIP(F16), anti-periostin antibody, rovalpituzumab soravtansine, ABT-414, IMGN289 AMG-595, brentuximab vedotin, iratumumab MDX-060, inotuzumab ozogamicin (CMC-544), pinatuzumab vedotin, epratuzumab SN38, polatuzumab vedotin, coltuximab ravtansine, SAR-3419, SGN-CD19A, indatuximab ravtansine, milatuzumab doxorubicin, IMGN-529, gemtuzumab ozogamicin, IMGN779, SGN CD33 A, and IGN523.

(42) A pharmaceutical composition comprising the conjugated antibody of any of the foregoing embodiments and one or more pharmaceutically acceptable carriers, diluents, or excipients.

(43) A method of treating solid cancer or liquid tumors comprising administering to a patient in need thereof an effective amount of a conjugated antibody, as recited in any of embodiments 1-41, or a pharmaceutical composition thereof, as recited in embodiment 42.

(44) The method of embodiment 43 for treating breast cancer, lung cancer, prostate cancer, skin cancer, colorectal cancer, bladder cancer, kidney cancer, liver cancer, thyroid cancer, endometrial cancer, muscle cancer, bone cancer, mesothelial cancer, vascular cancer, fibrous cancer, leukemia, or lymphoma.

(45) The conjugated antibody of any of embodiments 1-41for use in therapy.

(46) The conjugated antibody of any of embodiments 1-41 use in the treatment of solid cancers or liquid tumors.

(47) The conjugated antibody of embodiment 46 for use in the treatment of breast cancer, lung cancer, prostate cancer, skin cancer, colorectal cancer, bladder cancer, kidney cancer, liver cancer, thyroid cancer, endometrial cancer, muscle cancer, bone cancer, mesothelial cancer, vascular cancer, fibrous cancer, leukemia or lymphoma.

(48) Use of the antibody of any of embodiments 1-47 for manufacturing a medicament for the treatment of solid cancers or liquid tumors.

(49) The use of embodiment 48, wherein the solid cancers or liquid tumors are selected from breast cancer, lung cancer, prostate cancer, skin cancer, colorectal cancer, bladder cancer, kidney cancer, liver cancer, thyroid cancer, endometrial cancer, muscle cancer, bone cancer, mesothelial cancer, vascular cancer, fibrous cancer, leukemia, or lymphoma.

(50) A method for stimulating reactive oxygen species (ROS) production in a neutrophil, the method comprising contacting the neutrophil with the conjugated antibody of any of embodiments 1-41 under conditions whereby the conjugated antibody stimulates ROS production in the neutrophil.

(51) The method of embodiment 48, wherein the linker of the conjugated antibody comprises a polyethylene glycol spacer comprising at least 12 monomers.

(52) A compound having a formula R—P¹—P²—P³—NH—(CH₂CH₂O)ₙ—CH₂CH₂—Y or a salt thereof, wherein: R is a HC(═O)—; P¹ is Met(C(halogen)ₘ where m is 1-3 (e.g., Met(CF₃) or Met(CHF₂) or Met(CH₂F); P² is 1-6 proteinogenic or non-proteinogenic amino acids bonded to P¹ and to each other via peptide bonds; P³ is an amino acid comprising a side chain which comprises a —COOH moiety (e.g., glutamic acid or aspartic acid) or a —NH₂ moiety (e.g., lysine), optionally a glutamic acid residue connected through its side-chain gamma carboxyl group or a lysine residue connected through its side-chain epsilon amino group , and P³ is bonded to P² via a peptide bond; n is an integer selected from 3-24; and Y comprises amino or a cysteine reactive moiety, optionally wherein Y is selected from maleimide, maleimide-diaminopropionic, iodoacetamide, or vinyl sulfone.

(53) A compound having a formula R—P¹—P²—P³—NH—(CH₂CH₂O)ₙ—CH₂CH₂—Y or a salt thereof, wherein: R is a HC(═O)—; P¹ is Met or Met(C(halogen)ₘ where m is 1-3 (e.g., Met(CF₃) or Met(CHF₂) or Met(CH₂F); P² is 1-6 proteinogenic or non-proteinogenic amino acids bonded to P¹ and to each other via peptide bonds; P³ is an amino acid comprising a side chain which comprises a —COOH moiety (e.g., glutamic acid or aspartic acid) or a —NH₂ moiety (e.g., lysine), optionally a glutamic acid residue connected through its side-chain gamma carboxyl group or a lysine residue connected through its side-chain epsilon amino group, and P³ is bonded to P² via a peptide bond; n is an integer selected from 3-24; and Y comprises amino or a cysteine reactive moiety, optionally wherein Y is selected from maleimide, maleimide-diaminopropionic, iodoacetamide, or vinyl sulfone.

We claim:

1. A conjugated antibody or antigen-binding fragment thereof comprising an antibody or an antigen-binding fragment thereof that is conjugated to a peptide comprising an N-formyl-halogenated methionine residue at the N-terminus of the peptide.

2. The conjugated antibody of claim 1, wherein the N-formyl-halogenated methionine residue is N-formyl-trifluorinated methionine.

3. The conjugated antibody of claim 1, wherein the antibody is conjugated to the peptide via a linker.

4. The conjugated antibody of claim 3, wherein the conjugated antibody has a formula represented as:

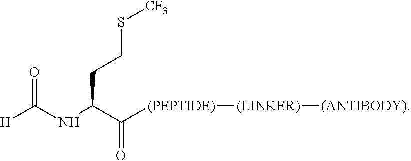

5. The conjugated antibody of claim 3, wherein the peptide comprises a C-terminal glutamic acid residue and the peptide is conjugated to the linker via an amide bond formed between the gamma carboxyl group of the glutamic acid and an amino group of the linker.

6. The conjugated antibody of claim 3, wherein the peptide comprises a C-terminal lysine residue and the peptide is conjugated to the linker via an amide bond formed between the epsilon amino group of the lysine and a carboxyl group of the linker.

7. The conjugated antibody of claim 3, wherein the conjugated antibody has a formula represented as:

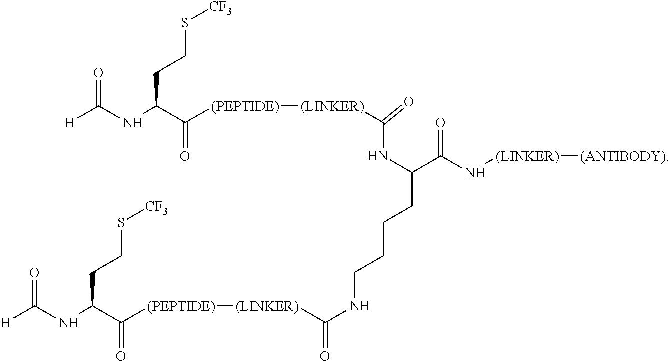

8. The conjugated antibody of claim 1, wherein the peptide comprises 2-10 amino acids.

9. The conjugated antibody of claim 1, wherein the peptide comprises 2-10 amino acids selected from D-amino acids.

10. The conjugated antibody of claim 1, wherein the peptide comprises 2-10 amino acids selected from homologues of amino acids lacking one or more methylene groups between the α-carbon and the side chain or homologous of amino acids possessing an additional methylene group between the α-carbon and the side chain.

11. The conjugated antibody of claim 10, wherein the peptide comprises 2-10 amino acids selected from homologues of amino acids lacking one or more methylene groups between the α-carbon and the side chain.

12. The conjugated antibody of claim 1, wherein the peptide comprises 2-10 amino acids selected from homo-amino acids.

13. The conjugated antibody of claim 1, wherein the peptide comprises 2-10 amino acids selected from bishomo-amino acids.

14. The conjugated antibody of claim 1, wherein the peptide comprises 2-10 amino acids selected from alkylated amino acids comprising an alkyl substitution on the α-carbon.

15. The conjugated antibody of claim 1, wherein the peptide comprises 2-10 amino acids selected from di-alkylated amino acids comprising a di-alkyl substitution on the α-carbon. 16, The conjugated antibody of claim 1, wherein the peptide comprises 2-10 amino acids selected from alkylated amino acids comprising an alkyl substitution on the amino group).

17. The conjugated antibody of claim 1, wherein the peptide comprises 2-10 amino acids selected from phenylalanine, tyrosine, tryptophan, histidine, proline, naphthylalanine.

18. The conjugated antibody of claim 1, wherein the peptide comprises 2-10 amino acids selected from nor-amino acids and linear core amino acids.

19. The conjugated antibody of claim 1, wherein the peptide comprises 2-10 amino acids selected from amino acids comprising a substitution selected from alkynyl, azido, thiophenyl, thienyl, pyridyl, anthrenyl, cycloalkyl, diphenyl, furyl, and naphthyl.

20. The conjugated antibody of claim 1, wherein the peptide comprises 2-10 amino acids selected from amino acids having a formula NH₂—CH₂—CH₂—(O—CH₂—CH₂)ₙ—COOH, wherein n is selected from 1-24.

21. The conjugated antibody of claim 1, wherein the peptide comprises 2-10 amino acids selected from β-amino acids, γ-amino acids, δ-amino acids, ε-amino acids, and ζ-amino acids.

22. The conjugated antibody of claim 1, wherein the peptide comprises 2-10 amino acids selected from cycloamino acids.

23. The conjugated antibody of claim 1, wherein the peptide comprises an amino-protecting group.

24. The conjugated antibody of claim 3, wherein the linker comprises a spacer arm having a length of about 10-50 angstroms.

25. The conjugated antibody of claim 3, wherein the linker comprises a spacer arm and the linker has a formula selected from:

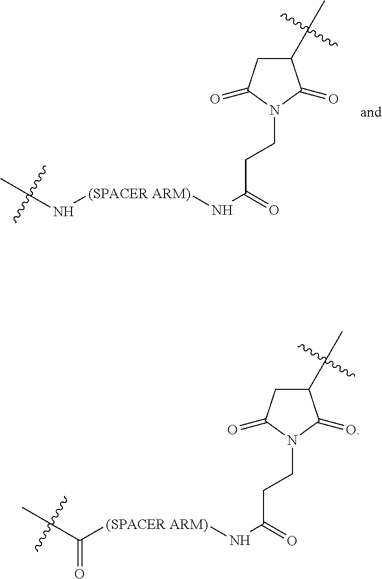

26. The conjugated antibody of claim 25, wherein the linker has a formula selected from 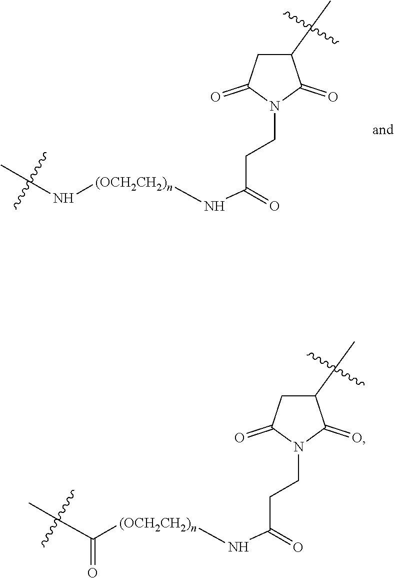
where n is an integer selected from 3-24.

27. The conjugated antibody of claim 24, wherein the spacer arm is a peptide sequence comprising amino acids selected from glycine, serine, and alanine.

28. The conjugated antibody of claim 3, wherein the linker comprises a polyethylene glycol (Peg) moiety (i.e., (—O—CH2-CH2)₁₋₂₄).

29. The conjugated antibody of claim 3, wherein the linker comprises a split polyethylene glycol moiety represented as -((Peg)₁₋₂₄)-(AA)₁₋₂-((Peg)₁₋₂₄)-, wherein AA is Glutamic acid residue connected through its side-chain gamma carboxyl group or Lysine residue connected through its side-chain epsilon amino group.

30. The conjugated antibody of claim 3, wherein the peptide-linker has a formula selected from: 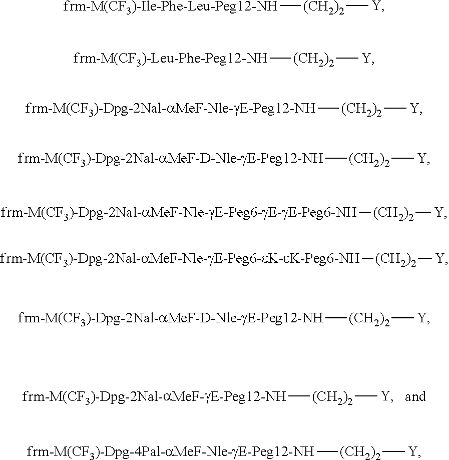
wherein Y comprises an amino group or a cysteine-reactive moiety for conjugating the peptide-linker to the antibody.

31. The conjugated antibody of claim 30, wherein the cysteine-reactive moiety is selected from maleimide, maleimide-diaminopropionic, iodoacetamide, or vinyl sulfone.

32. The conjugated antibody of claim 3, wherein the linker comprises a maleimide moiety and the linker is conjugated to the antibody via a thioether bond formed between the maleimide moiety and a cysteine residue of the antibody.

33. The conjugated antibody of claim 32, wherein the antibody comprises an IgG heavy chain constant region and light chain region wherein said cysteine residue is selected from residue 124 of the C_{H}1 domain, residue 378 of the C_{H}3 domain, or both of residue 124 of the C_{H}1 domain and residue 378 of the C_{H}3 domain.

34. The conjugated antibody of claim 1, wherein the antibody is a human antibody, a chimeric or hybrid antibody, or a humanized antibody.

35. The conjugated antibody of claim 1, wherein the antibody comprises an IgG heavy chain constant region selected from human IgGI isotype or human IgG4 isotype.

36. The conjugated antibody of claim 35, wherein the antibody comprises an IgG heavy chain constant region comprising an isoleucine substituted at residue 247, a glutamic acid substituted at residue 332, or both of an isoleucine substituted at residue 247 and a glutamic acid substituted at residue 332, with numbering based on EU Index Numbering.

37. The conjugated antibody of claim 36, wherein the antibody comprises a heavy chain constant region comprising the amino acid sequence of SEQ ID NO:12, 13, 14, 15, 16, 17, 18, 19, 20, 21, 52, 53, 54, 55, 56, or 57.

38. The conjugated antibody of claim 1, wherein the antibody is a monoclonal antibody.

39. The conjugated antibody of claim 1, wherein the antibody is a bispecific antibody.

40. The conjugated antibody of claim 1, wherein the antibody binds to an antigen selected from HER2, PSMA, TROP2, MUC-1, Nectin 4, LIV-1, mesothelin, MUC-16, folate receptor-R1, CEACAM5, GPNMB, CD56, STEAP1, ENPP3, guanylyl cyclase C, SLC44A4, NaPi2b, CD70, CA9 carbonic anhydrase, 5T4, SC-16, tissue factor, P-Cadherin, Fibronectin Extra-domain B, endothelin receptor ETB, VEGFR2, Tenascin c, periostin, DLL3, EGFR, CD30, CD22, CD79b, CD19, CD138, CD74, CD37, CD33, and CD98.

41. The conjugated antibody of claim 1, wherein the antibody comprises one or more of an HCDR1, an HCDR2, an HCDR3, an LCDR1, an LCDR2, and an LCDR3 of an antibody selected from T-DM1, ARX788, SYD985, MLN2704, PSMA-ADC, TACSTD2, sacituzumab govitecan, (IMMU-132), SAR-566658, enfortumab vedotin (ASG-22M6E), ASC-22CE, ZIP6, SGN-LIVIA, DMOT4039A, anetumab ravtansine (BAY-94-9343), BMS-986148, sofituzumab vedotin, mirvetuximab soravtansine (IMGN-853), vintafolide, labetuzumab SN-38, glembatumumab vedotin, lorvotuzumab mertansine (IMGN-901), vandortuzumab vedotin (RG-7450), AGS-16M8F, indusatumab vedotin (MLN-0264), ASG-5ME, lifastuzumab vedotin, TNFSF7, DNIB0600A, AMG-172, MDX-1243, vorsetuzumab mafodotin (SGN-75), BAY79-4620, TPBG, PF 06263507, SLTRK6 (ASG-15ME), anti-Fyn3, SC16LD6.5, HuMax-TF-ADC (TF-011-MMAE), PCA062, Human mAb L19, Human mAb F8, RG-7636, anti-VEGFR-2ScFv-As2O3-stealth nanoparticles, anti-TnC-A1 antibody SIP(F16), anti-periostin antibody, rovalpituzumab soravtansine, ABT-414, IMGN289 AMG-595, brentuximab vedotin, iratumumab MDX-060, inotuzumab ozogamicin (CMC-544), pinatuzumab vedotin, epratuzumab SN38, polatuzumab vedotin, coltuximab ravtansine, SAR-3419, SGN-CD19A, indatuximab ravtansine, milatuzumab doxorubicin, IMGN-529, gemtuzumab ozogamicin, IMGN779, SGN CD33 A, and IGN523.

42. A pharmaceutical composition comprising the conjugated antibody of claim 1 and one or more pharmaceutically acceptable carriers, diluents, or excipients.

43. A method of treating a solid cancer or liquid tumor comprising administering to a patient in need thereof an effective amount of a conjugated antibody, as recited in claim 1, or a pharmaceutical composition thereof, as recited in claim 42.

44. The method of claim 43 for treating breast cancer, lung cancer, prostate cancer, skin cancer, colorectal cancer, bladder cancer, kidney cancer, liver cancer, thyroid cancer, endometrial cancer, muscle cancer, bone cancer, mesothelial cancer, vascular cancer, fibrous cancer, leukemia, or lymphoma.

45. The conjugated antibody of claim 1 for use in a therapy.

46. The conjugated antibody of claim 1 for use in the treatment of a solid cancer or liquid tumor.

47. The conjugated antibody of claim 46 for use in the treatment of breast cancer, lung cancer, prostate cancer, skin cancer, colorectal cancer, bladder cancer, kidney cancer, liver cancer, thyroid cancer, endometrial cancer, muscle cancer, bone cancer, mesothelial cancer, vascular cancer, fibrous cancer, leukemia, or lymphoma.

48. Use of the antibody of claim 1 for manufacturing a medicament for the treatment of a solid cancer or liquid tumor.

49. The use of claim 48, wherein the solid cancer or liquid tumor is selected from breast cancer, lung cancer, prostate cancer, skin cancer, colorectal cancer, bladder cancer, kidney cancer, liver cancer, thyroid cancer, endometrial cancer, muscle cancer, bone cancer, mesothelial cancer, vascular cancer, fibrous cancer, leukemia, or lymphoma.

50. A method for stimulating reactive oxygen species (ROS) production in a neutrophil, the method comprising contacting the neutrophil with the conjugated antibody of claim 1 under conditions whereby the conjugated antibody stimulates ROS production in the neutrophil.

51. The method of claim 48, wherein the linker of the conjugated antibody comprises a polyethylene glycol spacer comprising at least 12 monomers.

52. A compound having a formula R—P¹—P²—P³—NH—(CH₂CH₂O)ₙ—CH₂CH₂—Y or a salt thereof, wherein:
R is a HC(═O)—;
P¹ is Met(C(halogen)ₘ where m is 1-3;
P² is 1-6 proteinogenic or non-proteinogenic amino acids bonded to P¹ and to each other via peptide bonds;
P³ is an amino acid comprising a side chain which comprises a —COOH moiety or a —NH₂ moiety and P³ is bonded to P² via a peptide bond;
n is an integer selected from 3-24; and
Y comprises amino or a cysteine reactive moiety.

53. A compound having a formula R—P¹—P²—P³—NH—(CH₂CH₂O)ₙ—CH₂CH₂—Y or a salt thereof, wherein:
R is a HC(═O)—;
P¹ is Met or Met(C(halogen)ₘ where m is 1-3;
P² is 1-6 proteinogenic or non-proteinogenic amino acids bonded to P¹ and to each other via peptide bonds;
P³ is an amino acid comprising a side chain which comprises a —COOH moiety and P³ is bonded to P² via a peptide bond;
n is an integer selected from 3-24; and
Y comprises amino or a cysteine reactive moiety.
